(19) United States
(12) Patent Application Publication
(10) Pub. No.: US 2013/0034594 A1
(43) Pub. Date: Feb. 7, 2013

(54) FIBERS CONTAINING FERRATES AND METHODS

(71) Applicants: Bruce F. Monzyk, Delaware, OH (US); Robert D. Giammar, Columbus, OH (US); Ada Cowan, Delaware, OH (US)

(72) Inventors: Bruce F. Monzyk, Delaware, OH (US); Robert D. Giammar, Columbus, OH (US); Ada Cowan, Delaware, OH (US)

(73) Assignee: BATTELLE MEMORIAL INSTITUTE, Columbus, OH (US)

(21) Appl. No.: 13/641,574

(22) PCT Filed: Apr. 21, 2011
(86) PCT No.: PCT/US11/33506

§ 371 Date: Oct. 16, 2012

Related U.S. Application Data
(60) Provisional application No. 61/326,438, filed on Apr. 21, 2010.

(51) Int. Cl. C09K 3/00; A01N 25/10; A01N 59/16; A01P 1/00; A47L 25/00; A61L 9/00; A61L 2/23; B08B 1/00; C02F 1/72; C11D 17/00; A01N 25/34

(52) U.S. Cl. 424/411; 252/186.1; 252/186.33; 252/175; 252/176; 510/367; 424/409; 424/646; 424/641; 423/210; 422/122; 134/6; 210/758; 15/209.1; 15/159.1

(57) ABSTRACT

A fiber containing ferrate, which comprises (1) one or more ferrate compounds, wherein the ferrate compound is selected from a group consisting of a metal ferrate(V) compound, metal ferrate(VI) compound, and a mixture thereof; and (2) one or more nonaqueous polymers. Also, methods are disclosed that make and/or use the fibers.

### BACKGROUND

Ferrate(VI), or “ferrate”, has long been known as a powerful oxidant that has been studied for potential uses in waste water treatment and in batteries. The literature also contains reports of the potential use of ferrate in certain organic oxidations, surface treatments, and blood clotting.

There are several ways of synthesizing ferrate. One such method is described by Johnson in U.S. Pat. No. 5,746,994 in which Fe³⁺ is oxidized with monoperoxide. Johnson reports that the isolation of potassium ferrate(VI) K₂FeO₄ in a sulfate matrix K₂SO₄ stabilizes the ferrate against decomposition and inhibits clumping from moisture adsorption. Johnson also mentions that potassium ferrate made by hypochlorite oxidation in a strongly alkaline solution and precipitated by the addition of KOH is stable indefinitely when kept dry.

An apparatus for synthesizing ferrate is described in U.S. Pat. Pub. No. 2005/0042155 A1.

Ferrate has been proposed for use commercially for water purification and its use in treating waste water has been discussed in scores of publications. For example, Deininger et al. in U.S. Pat. Nos. 4,983,306 and 5,380,443 has described treating water to remove metal ion contaminants, especially the transuranic elements. In this method, the pH of the water is adjusted to about 6.5 to about 14. Ferrate is especially useful for waste water treatment since it can remove a broad range of contaminants, disinfects many types of pathogens, and the iron(III) products coagulate and fall from solution, thereby also clarifying the water.

The use of ferrate in the presence of a phase transfer catalyst has been reported in oxidations of certain organic compounds. Song et al., in Huaxue Tongbao 69(3), 220-223 (2006) reported the conversion of benzyl alcohol to benzaldehyde by reaction with potassium ferrate in the cyclohexane/water in the presence of benzyltrimethylammonium chloride. Similar chemistry was described by Kim et al. in Synthesis, 10, 866-8 (1984).

Patterson in U.S. Pat. No. 6,521,265 describes a method of clotting blood by topically applying a ferrate paste to a wound. In this method, the compound is stored dry and unmixed and is mixed into a paste with the patient's blood or other aqueous media just prior to its application to a wound. Patterson states that the oxygen produced during the reaction substantially reduces the level of bacteria, virus and fungus at the wound. After treatment, the wound remains open unless the ferrate salt is combined with a bandage that has been impregnated or coated with a dry powder of one of the ferrate salt compositions. In this case, the impregnation is the same as merely coating the bandage with the dry powder of the ferrate salt. Patterson does not teach or suggest a way to disperse or embed the ferrate salt inside of the fiber composite of the bandage.

Thompson et al. in PCT Patent Application No. WO2008/151041 describe a method of using strontium ferrate(IV) to stop bleeding by spreading on the blood a mixture of the strontium ferrate powder with a polyacrylate polymer powder. Thompson et al. also state that such a mixture can be spread on top of, or coating the surface of, the bandage. However, before mixing the ferrate with the polymer, Thompson et al. teach that the ferrate has to be crushed, pulverized, and screened. Such a vigorous treatment can be applied to the stable lower oxidative states of ferrate compounds (IV, III, or II). It might cause premature decomposition of more reactive higher oxidative states of ferrate compounds, such as ferrate(V) and ferrate(VI) compounds.

Metal surfaces can be oxidized with a ferrate solution to form an oxide layer. Minevski et al. in U.S. Pat. No. 7,045,024 describe a process in which an aluminum surface is cleaned and then treated with a ferrate solution for a time ranging from about 1 second to about 5 minutes.

Champi et al. in U.S. Pat. No. 6,974,562 and U.S. Pat. Pub. No. 2005/0271575 describe methods of making ferrate immediately prior to use. This is advantageous since ferrate can degrade quickly in the presence of moisture. Champi et al. suggest that the ferrate could be encapsulated for future use in a membrane of molecular sieves, clay, porcelain, or other porous material that is not susceptible to oxidation. The membrane could be slightly water soluble so that the ferrate could be released over time. However, Champi et al. do not provide how to encapsulate the ferrate in the membrane, nor do Champi et al disclose that the ferrate can be a part of the membrane.

Champi et al. propose numerous uses for the ferrate, including: as an oxidant to prepare polymer and metal surfaces; removal of color from industrial electrolytic baths, synthesis of Fischer-Tropsch catalysts, purifying hemicellulose, as a selective oxidant in organic chemistry, disinfection as a biocide or virocide, phosphorylase inactivator in vitro, paint additive, denitration of flue gas, electrode, detoxifying cyanide from waste water, in cigarette filters, as an oxidant of pulp waste, removal of hydrogen sulfide, purifying waste water and drinking water, as an additive to cement as a structural hardener; as a disinfectant, removal of slime films such as in power plants and shipboard cooling systems, delignification of agricultural residues, magnetic filler for plastics, as a catalyst in burning coal, as a component of grinding wheels, in ceramic encapsulated rare earth ferrates where ferromagnetic properties are needed, removal of textile dyes from wastewater, treatment of boiler chemical cleaning wastes, oxidizing sulfur and cyanide containing compounds generated by oil refineries and coke processing plants, removing Mn from drinking water, removing As from drinking water, destroying chemical warfare agents, removing organic matter from drinking water, purifying water in a Jacuzzi or swimming pool and filtering away the resulting iron salts, cleaning waste water from animal and vegetable processing, treatment of any aqueous stream containing biosolids, radioactive cleanup, oxidizing pretreatment of chromium containing films, removing heavy metals from solution, cleaning or disinfecting metallic surfaces in medical devices or in the semi-conductor industry, disinfecting and cleaning instruments and surfaces for medical uses, and cleaning bilge water from ships.

Rainer et al. in U.S. Pat. No. 4,246,910 disclose a cigarette filter impregnated with alkali ferrates having an oxidative state of +4, +5, or +6. Specifically, the so called “impregnation” is accomplished by first blending K₂FeO₄ with a microporous polyethylene powder, and then placing the resulting mixture into a cigarette filter compartment. The resulting impregnated filter compartment contains a powdery mixture of the ferrate/polyethylene, in which polyethylene acts as a filler to spread out the ferrate, and prevent the pluggage of the filter by the resulting ferric oxide product after the ferrate has reacted with the contaminant. However, the filter has a disadvantage of being very dangerous to handle. Further, the ferrate would oxidize quickly upon exposure to moisture.

### BRIEF DESCRIPTION OF THE INVENTION

Broadly, the present invention provides a fiber containing ferrate, comprising (1) one or more ferrate compounds, wherein the ferrate compound is selected from a group consisting of a metal ferrate(V) compound, metal ferrate(VI) compound, and a mixture thereof; and (2) one or more nonaqueous polymers.

The word “nonaqueous” is defined as containing very little water, preferably contains no more than 3 wt % water. In other words, the nonaqueous polymer, the nonaqueous solvent, or nonaqueous excipients, should contain little or no water, preferably contains no more than 3 wt % water, more preferably contains no more than 1 wt % water, most preferably contains no more than 0.1 wt % water. The purpose of “containing little or no water” in nonaqueous polymer, solvent, and excipients is to prevent water from these components reach the ferrate compound to prematurely decompose the ferrate compounds. In other words, the “nonaqueous” polymer, “nonaqueous” solvent, and “nonaqueous” excipients contains little or no water so that the moisture in them do not cause any premature decomposition of the ferrate compounds during the process of making the ferrate fiber, or within the ferrate fiber.

Preferably, the metal ion of the ferrate compound is selected from a group consisting of an alkali metal, alkaline earth metal, an oxidation resistant transition metal from Groups IIB & IIIB, a Group (III)A metal, lanthanide metal, and a combination thereof. More preferably, the metal ion of the ferrate compound is selected from non-oxidizable oxidation states of Li, Na, K, Rb, Cs, Mg, Ca, Sr, Ba, Sc, Y, La, other lanthanide, Zn, Cd, Al, Ga, In, Tl, Pb(II), Bi(II), or mixtures thereof.

According to some embodiments of the present invention, the ferrate compound is encapsulated.

According to some embodiments of the present invention, the ferrate ion is embedded in a solid solution with one or more compatible ions. Preferably, the compatible ion comprises a sulfate ion, a chromate ion, a silicate ion, an aluminate ion, an orthophosphate ion, a borate ion, a carbonate ion, a titanate ion, a zirconate ion, a manganate ion, a molybdate ion, or a mixture thereof.

According to some embodiments of the present invention, the nonaqueous polymer comprises epoxy resin, alkyd, polyester, polyurethane, polyolefin, polyamide, polysulfide, polythiolether, phenolic polyether, polyurethane, polyvinyl, rosin polyesters, silicones, siloxanes, perfluorinated resin, other fluorinated resin, polytetrafluoroethylene (Teflon®), polyvinylidene difluoride, nylon and other polyamide, a copolymer thereof, or a mixture thereof.

According to some further embodiments, the fiber includes one or more nonaqueous solvents. Preferably, the nonaqueous solvent comprises xylene, toluene, petroleum distillate, ketone, N-methyl pyrrolidone, triethanolamine, 2-ethoxyethanol, other nonaqueous solvent, alcohol, or a mixture thereof.

According to some embodiments, the ferrate fiber further includes one or more excipients, preferably nonaqueous excipients. Preferably, the excipient comprises an amphiphilic material, a surfactant, a pH buffer, an alcohol, an encapsulation agent, a phase transfer catalyst, a wetting agent, a dispersant, a binder, a plasticizer, a gelling material, a caustic agent, a thickener, an accelerant, an emulsifier, an optional de-colorant, a humectants, an optional colorant, an optional antifungal, an optional antibacterial, or a mixture thereof. Preferably, the amphiphilic material comprises cationic surfactants, anionic surfactants, non-ionic surfactants, quaternary surfactants, amphoteric surfactants, zwitterionic surfactants, and combinations thereof.

In addition, the excipients comprises mono-phosphate, poly-phosphate, bicarbonate salt, calcium carbonate, sulfate salt, orthophosphate ester, orthophosphate salt, tetraorganoammonium ion, pyrophosphate salt, titanium dioxide, clay silicate, aluminum silicate, aluminate, aluminosilicate, talc, mica, silica, silicate, magnesium silicate, zinc oxide, barite sulfate, barium sulfate, or a mixture thereof.

Preferably, the ferrate compound is present in a concentration that does not interfere with the mechanical integrity of the fiber. More preferably, the ferrate compound is present in a concentration of up to about 25 v/v %.

Alternatively, the present invention provides a method of making a fiber containing ferrate, which comprises

a. providing a nonaqueous formulation of one or more ferrate compounds, wherein the ferrate compound is selected from a group consisting of metal ferrate(V) compounds, metal ferrate(VI) compounds, or mixtures thereof;

b. providing one or more nonaqueous polymers; and

c. dispersing the ferrate formulation of step (a) in the nonaqueous polymer to produce the fiber.

Preferably, the step (d) is accomplished by means of electric field effect technology.

Preferably, a metal of the ferrate compound is selected from comprises Li, Na, K, Rb, Cs, Mg, Ca, Sr, Ba, Sc, Y, La, other lanthanide, Zn, Cd, Al, Ga, In, Tl, Pb, Bi, or a mixture thereof

According to some embodiments of the present invention, the ferrate formulation of step (a) comprises one or more nonaqueous solvents, and/or one or more excipients.

Preferably, the excipient comprises an amphiphilic material, a surfactant, a pH buffer, an alcohol, an encapsulation agent, a phase transfer catalyst, a wetting agent, a dispersant, a binder, a plasticizer, a gelling material, a caustic agent, a thickener, an accelerant, an emulsifier, an optional de-colorant, a humectant, an optional colorant, an optional antifungal, an optional antibacterial, or a mixture thereof.

According to some embodiments, the ferrate compound formulation comprises one or more encapsulated ferrate compounds.

According to some embodiments, a ferrate ion of the ferrate compound is embedded in a solid solution with one or more compatible ions. Preferably, the compatible ion comprises a sulfate ion, a chromate ion, a silicate ion, an aluminate ion, an orthophosphate ion, a borate ion, a carbonate ion, a titanate ion, a zirconate ion, a manganate ion, a molybdate ion, or a mixture thereof.

According to some embodiments, the nonaqueous polymer comprises epoxy resin, alkyd, polyester, polyurethane, polyolefin, polyamide, polysulfide, polythiolether, phenolic polyether, polyurethane, polyvinyl, rosin polyesters, silicones, siloxanes, perfluorinated resin, other fluorinated resin, polytetrafluoroethylene (Teflon®), polyvinylidene difluoride, nylon and other polyamide, a copolymer thereof, or a mixture thereof.

In some alternative embodiments, the present invention provides a method of cleaning and/or disinfecting using a pad or a brush composed of fibers containing ferrate of the embodiments described above. These ferrate fibers can also be used in a method of treating air or water.

In some alternative embodiments, the present invention provides a pad or a brush suitable for cleaning or disinfection, comprising fibers containing ferrate of the above described embodiments.

In some alternative embodiments, the present invention provides a filter suitable for air or water treatment, comprising fibers containing ferrate of the above described embodiments.

### BRIEF DESCRIPTION OF THE DRAWINGS

FIG. 1 is a schematic diagram that illustrates a typical process of embedding ferrates (ferrate compounds) in ferrate fibers according to the invention.

FIGS. 2A through 2F are schematic diagrams that illustrate various nozzle configurations according to the invention.

FIG. 3 illustrates a typical apparatus for spraying with a concentric nozzle using an electric charge or electric field to produce ferrate fibers of the present invention.

FIG. 4 illustrates several embodiments for collecting ferrate fiber products according to the invention.

FIGS. 5A and 5B are schematic diagrams that illustrate apparatus for producing a mat containing ferrate particles or compounds according to another aspect of the invention.

FIGS. 6A and 6B are schematic diagrams that illustrate an apparatus with a plurality of circular edges for continuous production of ferrate fiber materials according to another aspect of the invention.

FIGS. 7A and 7B are schematic diagrams that illustrate an apparatus for having a double belt for continuous production of ferrate fiber materials according to yet another aspect of the invention.

FIGS. 8A and 8B are schematic diagrams that illustrate an apparatus for the formation and/or collection of the ferrate fiber materials of the present invention, having a rotating plate or disc with one or more openings that define an edge.

FIG. 9 is a schematic diagram illustrating opposing nozzle configurations according to the invention.

FIG. 10 illustrates a typical apparatus for spraying with a concentric nozzle with two liquid reservoirs using an electric charge or electric field.

FIG. 11 illustrates a typical apparatus for spraying with a Siamese type nozzle using an electric charge or electric field.

FIG. 12 illustrates a typical apparatus for spraying with a Gemini type nozzle using an electric charge or electric field.

### DETAILED DESCRIPTION OF THE INVENTION

Broadly, the present invention is about fibers containing ferrate(s), and methods of making and using such fibers (also called “the ferrate fiber” or “the fiber”). The fibers can be continuous, short, or micro-sized. The ferrate fiber is actually the ferrates filled fibers, or fibers with ferrates embedded in them. The ferrates can be fully embedded or partially embedded in the fiber. That is, in some embodiments, parts and/or all of some ferrate compounds protrude out of the ferrate fiber, while most of the ferrate compounds are embedded in the ferrate fiber. Such fibers can be made into pads, brushes, or filters of all sizes, which can then be used to clean, disinfect, and/or decontaminate any surfaces, air, and/or water. Surfaces suitable for cleaning by the ferrate fibers include marble and steel surfaces that can be eroded by other cleaners

Preferably, the fibers of the present invention comprise (a) one or more ferrate compounds, wherein the ferrate compounds comprise metal ferrate(V) compounds, metal ferrate(VI) compounds, or mixtures thereof; and (b) one or more nonaqueous polymers. The “nonaqueous polymer” is defined in this application as a polymer containing very little water, preferably contains no more than 3 wt % water. The nonaqueous polymer, in the context of this application, can dissolve in an aqueous solvent, or in a nonaqueous solvent, or both. The “aqueous solvent” is defined in this application to be interchangeable with “water.” The “nonaqueous solvent” is defined as any solvent other than water, preferably containing no more than 3 wt % water. Some of the solvents can be treated with dehydrating agents to reduce their water or moisture content to be no more than 3 wt % to be used as “nonaqueous solvents” in the present application.

The word “nonaqueous” in this application is defined as “containing little or no water,” preferably contains no more than 3 wt % water. In other words, the nonaqueous polymer, the nonaqueous solvent, or nonaqueous excipients, should contain little or no water, preferably contains no more than 3 wt % water, more preferably contains no more than 1 wt % water, most preferably contains no more than 0.1 wt % water. The purpose of “containing little or no water” in nonaqueous polymer, solvent, and excipients is to prevent water from these components reach the ferrate compound to prematurely decompose the ferrate compounds during the process of making the fiber or within the fiber. In other words, the “nonaqueous” polymer, “nonaqueous” solvent, and “nonaqueous” excipients contains little or no water so that the moisture in them do not cause any premature decomposition of the ferrate compounds during the process of making the ferrate fiber, or within the ferrate fiber.

The metal ferrate(V) compound and the metal ferrate(VI) compound are collectively called the ferrate compound, the ferrate, or the metal ferrate compound. As used herein, “ferrate” does not refer to just any compound that contains iron, a negatively charged molecule. For example, hexacyanoferrate(II) is not “ferrate” in the description herein. The composition/formulation containing the ferrate can also be called the ferrate formulation. It is known that the ferrate compound may contain a metal ion and a ferrate ion. As used herein, the “ferrate ion” is referring to the ferrate(V) molecular ion and/or ferrate(VI) molecular ion.

Upon reacting with oxidizable water, organics, inorganics, or metal (or through other types of oxidative reaction), the ferrate(VI) anion (“Fe(VI)”) is reduced to the lower oxidation state of the ferrate(V) anion (“Fe(V)”) or the ferrate(IV) anion (“Fe(IV)”). Fe(IV) is still a relatively strong oxidant, and so it can then be further reduced to more stable and lower oxidative states of Fe(III) or Fe(II). Fe(III) oxide, oxyhydroxide, hydroxide with phosphates and other compounds, are usually referred to collectively as “ferric” oxides and ferric phosphates. Fe(III) is only slightly oxidatively reactive. Ferrate(II) is water soluble, and it is a strong reducing agent. Being very water soluble and only reductively reactive, ferrate(II) is not very useful for cleaning and/or disinfection. Further, it reacts quickly with O₂ from the air to form a flocculant, which is not a desirable result in cleaning or decontamination. However, being oxidation/reaction products of the ferrate of the present invention, ferrate(II) and (III) are very safe and easily disposed of without creating any environmental contamination problem.

The fiber of the present invention is capable of incorporating the ferrate within itself without creating any substantial incompatibility issue with organic components in the fiber. We have discovered that this new and unexpected result is possible so long as either the potassium ferrate is insoluble in the fiber material when in liquid form, or that the fiber material is very slow or “inert” to react at room temperature (preferably, it is resistant to oxidation by the ferrate). It is also possible when the fiber absorbs H₂O so strongly that it prevents water from reaching the ferrate crystals suspended within the fiber matrix. At the same time, the ferrate in the fiber retains its oxidative capabilities for extended periods, even for months, enabling the fiber to be used for purposes of cleaning, disinfection, and the like. Preferably, the metal cation in the ferrate compound for the fiber of the present invention is an alkali metal, alkaline earth metal, an oxidation resistant transition metal from Groups IIIB or IIB, a Group (III)A metal, a lanthanide metal, and/or a combination thereof. The use of the designation “Group” in this context refers to the Chemical Abstracts Service version of the Periodic Table of the Elements and are same as the “New” notation Groups 1, 2, 3, 12, 13, 14 and 15, as well as the Lanthanides. Unlimited examples of the metal cation are nonoxidizable oxidation states of Li, Na, K, Rb, Cs, Mg, Ca, Sr, Ba, Sc, Y, La, other lanthanide, Zn, Cd, Al, Ga, In, Tl, Pb(II), Bi(III), or combinations thereof.

According to some embodiments of the present invention, the ferrate fiber of the present invention can be formed into (1) brush or pad or any other means of abrasion or scrubbing action; and (2) gas or liquid filter. When in use, the rubbing action of the brush/pad against a surface would release minor amounts of calcite (or other co-filler excipients/minerals) and release the ferrate (ferrate(V) and/or ferrate(VI)), allowing the ferrate to perform disinfection or oxidative cleaning functions. The brush or pad can erode through use, or it can be non-eroding even after use. The erosion property of the brush/pad can be controlled by selecting an appropriate matrix polymer or a combination of the polymers suitable for making the fiber. For example, nylon or polyurethane matrix material can be used when a slow wear/non-eroding fiber is desirable, while polyolefin-based matrices, such as polyethylene, polyacrylate, and polypropylene, is used where an eroding fiber is desirable to release ferrate more rapidly. Polyesters and polyethers have an intermediate hardness and wear resistance. The brushes/pads are useful for cleaning surfaces, including porous surfaces, of metals, ceramics, asphalt tiles, concrete, plaster, brick, stucco, roofing shingles, glass, painted surfaces, composites, including fiberglass and carbon fiber composites, wood of all types, marble (where the ferrate fiber would not erode this mineral as does many other known cleaners), clothes (woven and unwoven), and the like.

The filter can be used in either an air or a liquid/aqueous environment. When the filter is used to clean/purify air, the contaminants might migrate through the interspatial passages formed by the organized or disorganized layering of the ferrate fiber, and then become trapped within the fiber matrix. At the same time, it also introduces moisture into the fiber via adsorption, and this moisture becomes captured on the surfaces and pores of fiber etc. Once captured, moisture penetrates to the ferrate crystals in the fibers, including both the ferrate crystals located on the surface of the fiber and embedded within the fibers. The moisture then dissolves the ferrate into the moist liquid film or liquid-filled pore, in which the ferrate can react with, and thereby “destroy,” the contaminants or kill/disinfect biological micro-organisms trapped within the fiber. In a liquid environment, the ferrate can be released in a controlled fashion to react with contaminants.

In order for the ferrate in the fiber to perform its cleaning/disinfection function, free ferrate ions must be released as needed or in a controlled fashion to reach the target pathogenic microorganisms, toxic or odorous molecules, soil substances, and the like. The controlled release of the ferrate in the fiber can be achieved through five factors: (1) the solubility of the ferrate compound or formulation; (2) the hygroscopicity of the fiber and/or the ferrate formulation; (3) pH control; (4) physical/mechanical abrasion exposing the embedded ferrate compound or formulation, and (5) the porosity of the fiber. These five factors are inter-related. For example, the hygroscopicity of the ferrate formulation can change the porosity of the fiber, and pH variation can change the solubility of the ferrate.

Specifically, the higher the solubility of the ferrate, the more free ferrate ions can be released upon exposure to the moisture. The higher the hygroscopicity of the ferrate formulation and/or the ferrate fiber, the more likely the ferrate crystals are exposed to the moisture in the fiber. The more porous the ferrate fiber is, the more likely the ferrate can be exposed to the moisture/water. The lower the pH of the ferrate/ferrate formulation; the higher the solubility and reactivity of the ferrate. In addition, the higher the hygroscopicity of the ferrate fiber, the more likely that the fiber might swell upon absorption of moisture to become more porous, which in turn, might provide more paths for the moisture to reach the ferrate, or for the freed ferrate to migrate to the moisture. As the result, the ferrate is discharged from the fiber (or brushes/pads/filters made of the ferrate fiber) to react with the surface/environment to be cleaned. Of course, the more the fiber is physically brushed or rubbed, the more likely the embedded ferrate crystals are exposed to moistures and/or contaminants.

Other factors include temperature, the shapes of the ferrate crystals, the aspect ratios of the ferrate crystals, the positioning of the ferrate crystals inside the fiber, which can also impact one or more of the above five factors. For example, the shape of the ferrate crystals can influence the solubility of the ferrate. Further, the shapes of the ferrate crystals might be used to control the porosity of the ferrate fiber. In some embodiments, for purposes of having a faster or more immediate release of ferrate, the ferrate crystals are preferably embedded throughout the fiber and are placed so that the ferrate crystals are in physical contact with each other. For example, if a ferrate crystal A is in physical touch with a ferrate crystal B. After the ferrate crystal A is exposed to moisture, leached out, and reacted with the target microbes/chemical/contaminant(s), it leaves an empty space/pore, which then enables the moisture to reach the ferrate crystal B right next to the reacted ferrate crystal A space to release the ferrate ion from the ferrate crystal B. In other words, the shape, length, and the positioning of the ferrate crystals can create or increase the porosity of the fiber for the moisture to reach the ferrate crystals at a faster rate, delivering more free ferrate ions per any given volume of the ferrate fiber.

The greater the aspect ratio of a ferrate crystal, the more likely these ferrate crystals will touch each other physically at a lower loading percentage in the fiber. As such, the aspect ratio of a ferrate crystal can also control the rate of the release of the ferrate ion in the fiber. In some cases, the aspect ratio of a ferrate crystal is determined by its oxidative state. For example, sodium ferrate(V) compounds are usually in the shape of a long needle with a high aspect ratio, while potassium ferrate(VI) compounds have a more platelet or rhombic shape with a lower aspect ratio when compared to the ferrate(V) compound. Similarly, barium ferrate(VI) and calcium ferrate(VI) have small aspect ratios near 1 and a very small particle size. On the other hand, barium ferrate(VI) and strontium ferrate(VI) have a very high surface area volume unit of crystal. The long needle shape of the ferrate(V) compounds have a longer reach, up to at least 100 microns, which enables the ferrate(V) compound crystals to be in physical touch of each other at a lower loading volume percentage of the ferrate. Therefore, if a faster release of the ferrate ion is desired, the sodium ferrate(V) compound can be used instead of the potassium ferrate(VI) compound. In other cases, the ferrate(V) compounds can be used in conjunction with the ferrate(VI) compound to obtain variable rates of release of the ferrate ions. Barium ferrate(VI) and barium ferrate(V) have very low solubility so they can be used as the slow release ferrate compounds. In addition, using solid solution crystals of the ferrate and compatible ions, for example solid solutions of potassium ferrate and potassium sulfate, is another means to reduce the ferrate release rate where only very small amounts (e.g. 0.1-11 ppm) would be released over an extended period of time (e.g. in air filtration or water purification at the point of use, and the like).

All of the above factors discussed above are dependent upon the solubility of the ferrate compound to release the ferrate ion in controlling the reactivity of the ferrate. In the present invention, the solubility of the ferrate can first be controlled by the metal cation of the ferrate compound. The preferred metal ion for achieving the slower release of the ferrate ion from the ferrate compound is alkaline earth metal ion, such as strontium or barium. Such alkaline earth metal ions stabilize ferrate anions through forming salts of low solubility in both water and organic phase and enable them to exist in a very rare high oxidative state of Fe(IV), Fe(V), or Fe(VI). Specifically, alkaline earth metal ions, along with other metal ions mentioned above, can produce ferrate compounds with a low solubility in water in the range of about 0.001 ppm to about 2000 ppm at a temperature in the range of about 0° C. to at least 71° C., and sometimes to about 100 ° C.

On the other hand, the alkali metal ions form ferrate salts of relatively higher solubility in aqueous phases (as would be present in aqueous scrubbing) and moisture films (as would be present in air filters), while at the same time, the alkali metal ions enable the resulting iron salt to exist in a high oxidative state of Fe(V) or Fe(VI). The ferrate compounds with higher solubility can be used for immediate release of the ferrate ion, while the ferrate compounds of lower solubility can be used to release the free ferrate ions over time. Further, mixtures of ferrate compounds of different solubility can be co-dispersed/co-mixed inside of the fiber to achieve both immediate and extended release of the free ferrate ions upon use. This variable controlled release of the ferrate ions is very useful for both brushing action and the filtering uses.

The solubility of the ferrate compound can also be controlled by encapsulation and by placing the ferrate in a solid solution, such as ferrate doped potassium sulfate or potassium chromate(VI). The ferrate compound with a higher solubility can be encapsulated to control and/or reduce the release rate of the free ferrate ions. The encapsulation can be porous, allowing certain amount of moisture to permeate through to the ferrate compound to release the ferrate ions in a slower fashion. Such porous encapsulation can be accomplished by encapsulating the ferrate into a zeolite, aluminate, zircoaluminate, and the like. The encapsulation can also be nonporous, having little or essentially no permeability to moisture, liquid or vapor. This type of encapsulation can be done by encapsulating the ferrate with silica or potassium orthophosphate, or overgrowing the ferrate crystal with potassium sulfate. The nonporous encapsulation can enhance the stability of the ferrate compound of any solubility, especially that of higher solubility, to enable the ferrate to be compatible with other components of the ferrate formulation and to be compatible with the polymer in the fiber.

The nonporous encapsulation of the ferrate preferably has a hydrophobic coating or wall composed of hydrophobic excipients or materials. Inside of the microcapsules, one or more hygroscopic compounds or solvents can be included, in which the ferrate is substantially not soluble. The hygroscopic compounds absorb moistures inside themselves and away from the ferrate. This type of encapsulation is similar to a sealed chamber containing desiccants, in which the desiccants are hygroscopic and absorb the moisture away from the environment in the chamber, and thus keeping the moisture low in the sealed chamber.

The encapsulation process also helps control the rate of ferrate release/reactivity in the scrubbing or filtering application. For examples, filters are often used for months between change/replacement; during that time, a large quantity of micro-organisms, as live cells, cysts, spores, viruses, and the like, would accumulate, making the filter material a biohazard or a chemical hazard. Since only about 0.1-10 mg/L (ppm) of the ferrate is needed to disinfect these organisms, a low rate of ferrate release/activity desired or is needed for the filter to be self-disinfecting or self-sterilizing, possibly extending the service life for the filters made of the ferrate fiber by many days, months, or even years.

Alternatively, or in combination with the encapsulation, ferrate ions of the ferrate compounds can be incorporated into solid solution crystals of low solubility with other compatible ions. The solid solution crystals can be made by the process of diffusion and/or absorption from aqueous solutions, sprays with tumbling, co-precipitation/co-crystallization, and the other acceptable techniques. Suitable compatible ions can include, but are not limited to, neutral or pH basic clays, minerals, low soluble salts, talcs, glass fibers (pH adjusted), silicates, inerts such as gypsum, sodium sulfate, and the like. Such formulated solids can reduce the rate of release of free ferrate ions in a controlled fashion because the bulk solid is very slow to dissolve, slow to leach in thin adsorbed moisture films, or it can be substantially insoluble. For enhanced control a selected amount of ferrate ions can be embedded in solid solution crystals through crystallization or ion exchange processes already known in the art. After incorporating the solid solution or formulation of the ferrate compound into the fiber, the solid solution crystals can act as filler carrier salts in carrying the ferrate ions in the fiber. As a carrier, the solid solution can facilitate the even spreading of the ferrate ion in the fiber even when there is a very a low concentration of the ferrate in the fiber. As such, the solid solution method can control the rate of release of ferrate ions in the fiber to perform cleaning/disinfecting functions; while at the same time, it can prevent spontaneous premature/useless decomposition of the ferrate ions.

The compatible ion can include, but is not limited to, a sulfate ion, a chromate ion, a silicate ion, an aluminate ion, an orthophosphate ion, a borate ion, a carbonate ion, a titanate ion, a zirconate ion, a manganate ion, a molybdate ion, or a mixture thereof.

In addition to, or in combination with, the solubility of the ferrate compound, and the hygroscopicity of the compatible nonaqueous polymers and the ferrate formulations can be used to control the release of the ferrate ions. A preferred ferrate formulation may optionally include one or more nonaqueous solvents, one or more excipients, preferably nonaqueous excipients, or a combination thereof. The “nonaqueous polymer” is defined in this application as a polymer containing very little water, preferably contains no more than 3 wt % water. The nonaqueous polymer, in the context of this application, can dissolve in an aqueous solvent, or in a nonaqueous solvent, or both.

Preferably, the suitable or compatible nonaqueous polymer can be, but is not limited to, epoxy resin, alkyd, polyester, polyurethane, polyolefin, polyamide, polysulfide, polythioether, phenolic polyether, polyurethane, polyvinyl, rosins, polyesters, silicones, siloxanes, perfluorinated resin, other fluorinated resins, polytetrafluoroethylene (Teflon®), polyvinylidene difluoride, nylons and other polyamides, copolymers thereof, blends, or mixtures thereof. Some of the polymers may be somewhat hygroscopic. The hygroscopic polymers can absorb and retain a certain amount of water to prevent the moisture in the air from reaching the ferrate to prematurely decompose the ferrate ions. Hydroscopic polymers are nylons, other polyamides, polyurethanes, polyvinylalcohols, polyethers, cellulosics, silicones, and the like.

Compatible/nonreactive, nonaqueous and yet volatile solvents are preferred in the present invention. The nonaqueous solvents (also called the nonaqueous plasticizer) are defined as any solvents other than water, preferably contain no more than 3 wt % water. Some of the solvents can be treated with dehydrating agents to reduce their water or moisture content to be no more than 3 wt % to be used as “nonaqueous solvents” in the present application. The suitable solvents are useful in that they can quickly evaporate in part or in full, leaving the ferrate fiber porous to highly porous depending on the volume percentage of the solvents incorporated in producing the original ferrate-filled fiber. Such solvents can be halogenated hydrocarbons, such as carbon tetrachloride, methylene chloride, freons, hydrocarbons such as petroleum ethers, liquid propane, pentane, butane, hexane, and the like, and liquid aromatic hydrocarbons such as xylene, toluene, benzene, and the like.

In addition, the nonaqueous solvents can be used to reduce viscosity and hardness of the polymers, retain and prevent moisture from reaching the ferrate prematurely, and/or slow down the thickening or crosslinking reaction of the polymer mixture. The unlimited examples of the nonaqueous solvent are xylene, toluene, petroleum distillate, ketone, carboxylic acid ester, N-methyl pyrrolidone, triethanolamine, 2-ethoxyethanol, soy oil ester, alcohol such as n-butanol, iso-propanol, t-butyl ether, iso-butylether, t-amyl alcohol, other nonaqueous solvent, or combinations thereof. Preferably, the nonaqueous solvent is strongly resistant to oxidation by the ferrate. In case that the nonaqueous solvent is only weakly or somewhat resistant to oxidation by the ferrate (which is also called the weakly oxidative resistant solvent), the ferrate is preferably substantially insoluble in the solvent. Alternatively, the ferrate can be encapsulated or put into a solid solution so that the encapsulated ferrate or ferrate solid solution is substantially insoluble in the weakly oxidative resistant solvent.

Among the nonaqueous solvents, monomeric, oligomeric and/or polymeric alcohols or ether alcohols, collectively called “alcohols,” can be used to keep moisture away from the ferrate compounds, either as a residue in the fiber or during the making of the ferrate fiber. As such, the alcohols can be used to increase hygroscopicity of the fiber or the ferrate formulation to prevent premature dissolution and decomposition of the ferrate ions, or to extend the stability and shelf life of the ferrate in storage. In the present invention, the preferred ferrates are substantially insoluble in the preferred alcohols. For ferrates that are soluble in the alcohols, they can be encapsulated and placed into a solid solution so as to be substantially insoluble in the alcohols. In some cases, the alcohols can absorb and retain up to at least 5 wt % of water while preventing excessive dissolution of the ferrate in the fiber. This combination is highly unexpected because the ferrate is known to rapidly oxidize alcohols when the ferrate is dissolved in an aqueous solution.

In some embodiments, the fiber of the present invention may include one or more excipients, whose hygroscopicity may be selected to control the release of the ferrate ion. In addition, the excipients are often added to produce a fiber of desired physical properties, such as strength (as measured by stress-strain curves known as “toughness”), flexibility (flexural modulus), hardness control, abrasiveness, impact resistance, electrical conductivity, static electricity discharge, or shrinkage control, etc. The excipient can be, but is not limited to, an amphiphilic material, a surfactant, a pH buffer, an encapsulation agent, a phase transfer catalyst, a wetting agent, a binder, a dispersant, a gelling material, a caustic agent, a thickener, an accelerant, an emulsifier, an optional de-colorant, a humectant, an optional colorant, an optional antifungal or mildew-cide, an optional antibacterial, or a mixture thereof.

Unlimited examples of the preferred excipients include mono-phosphates, poly-phosphates, bicarbonate salts, calcium carbonates, sulfate salts, orthophosphate esters, orthophosphate salts, symmetric or asymmetric tetraorganoammonium ions, pyrophosphate salts, titanium dioxides, clay silicates, aluminum silicates, aluminates, aluminosilicates, talc, mica, silica, silicates, magnesium silicates, zinc oxides, barite sulfates, barium sulfates, or mixtures thereof. Some of these examples can have multiple functions. For example, mono-phosphates, carbonates, silicates, borates, pyrophosphates, or poly-phosphates can be used as pH buffers to control ferrate ion reactivity, as compatible ions in a solid solution with ferrate ions, or they can be used as encapsulation agents.

As needed in some situations, some excipients, such as phase transfer catalysts, can assist freed ferrate ions in migrating longer distances or faster to the site of use for cleaning. Further, they can help the freed ferrate ions penetrate the space and interior of hydrophobic contaminants, and other purposes. Other excipients, such as KOH or NaOH, having a very high hygroscopicity, can be used to increase the reactivity of the ferrate by providing moisture to the ferrate in a low moisture environment, such as in a desert-like environment. In a normal environment, KOH or NaOH can act as a solvent or “catalyst” for ferrate activity because it can absorb moisture from the environment quickly and in a huge amount.

In some embodiments, the excipients, such as pH buffers, can be used to control pH of the ferrate formulation or the fiber. A pH buffer, such as calcium carbonate, or the calcium or other salts of mono-phosphates, carbonates, silicates, borates, pyrophosphates, or poly-phosphates, can be used to control the reactivity of free ferrate ions inside or released by the fiber, and thus, it can control the response time, compatibility and the oxidation/cleaning/disinfection capacity of the fiber. To maintain stability and to reduce the reactivity of the ferrate in the formulation or the fiber for a long term field use, a pH buffer might be needed to maintain the pH of the formulation to be at least 8 or above, preferably at 11 or above, and more preferably at 12 or above as measured by a slurry of the ferrate fiber in water. Conversely, the pH of the ferrate formulation and/or the ferrate fiber can be reduced to increase the rate of release of the ferrate ions and to increase the reactivity of free ferrate ions. For this control the pH buffers are selected to maintain the pH<9 and as low as ˜4, and these are preferred for “Brillo Pad” type applications. Lower pHs tend to cause the ferrate to react more quickly, or even immediately, and so are useful when the situation requires immediate actions from the ferrate fiber.

In some embodiments, an amphiphilic material can be added as an excipient to function as a surfactant to reduce the surface and interfacial tension between two immiscible liquids, such as the liquid ferrate formulation and the liquid polymer during the making of the ferrate fiber. Such an amphiphilic material can be used to facilitate the production of the fiber by means of electric field effect technology (EFET), which will be discussed in more details below. The amphiphilic material can be selected from cationic surfactants, anionic surfactants, non-ionic surfactants, quaternary surfactants, amphoteric surfactants, zwitterionic surfactants, and mixtures thereof.

The unlimited examples of the preferred anionic surfactant are petroleum sulfonate, lauryl sulfonate, lauryl sulfate, C8 to C18 hydrocarbon sulfonates and sulfates, and the like, non-ionic surfactants, quaternary surfactants, amphoteric surfactants, zwitterionic surfactants, and combinations thereof. The preferred cationic surfactant can be an alkylamine having a carbon chain length of between 8 and 18 (including unsaturated carbon chains such as an oleyl group), an alkoxylated amine having between 8 an 18 carbon atoms, such as Ethomeen®0 SI12 (bis(2-hydroxylethyl)soyaalkylamine), Ethomeen® SI15 (polyoxyethylene (5) soyaalkylamine), and Ethomeen® S/25 (polyoxyethylene (15) soyaalkylamine), which are available from Akzo Nobel Surface Chemistry LLC, Chicago, Ill., and combinations thereof. The preferred non-ionic surfactant can be a polyoxyethylene alcohol such as Brij™ 93 (polyoxyethylene (2) oleyl ether) or Brij™ 97 (polyoxyethylene (10) oleyl ether), a polyoxyethylene sorbitan fatty acid ester such as Tween™ 80 (polyoxyethylene (20) sorbitan monooleate), and combinations thereof, which are available from Uniqema, New Castle, Del., acetylenic and ethoxylated acetylene diol surfactants such as SurfynoFM 104, SurfynoFM 420,

SurfYnoFM 440, etc., which are available from Air Products and Chemicals, Inc. of Allentown, Pa., and combinations thereof. In addition, the amphiphilic material can include alkylamines, alkylamine ethoxylates, alkylamine propoxylates, alkylamine propoxylates-ethoxylates, fatty alcohol ethoxylates, fatty alcohol propoxylates, fatty alcohol propoxylates-ethoxylates, fatty acid ethoxylates, fatty acid propoxylates, fatty acid propoxylates-ethoxylates, synthetic long-chain alcohol ethoxylates, synthetic long-chain alcohol propoxylates, synthetic long-chain alcohol propoxylates-ethoxylates, synthetic long-chain acid ethoxylates, synthetic long-chain acid propoxylates, synthetic long-chain acid propoxylates-ethoxylates, alkylphenol ethoxylates, alkylphenol propoxylates, alkylphenol propoxylates-ethoxylates, alkylpolyglucosides, sorbitol esters, sorbitan esters, sorbitol ester ethoxylates, sorbitan ester ethoxylates, polyoxypropylene-polyoxyethylene block copolymers (e.g., BASF's “PLURONICS™”), ethylenediamine-polyoxypropylene-polyoxyethylene block copolymers (e.g., BASF's “TETRONICS™”+), and mixtures thereof.

The amount of ferrate(V) and/or ferrate(VI) (the ferrate) in a formulation of the invention can vary so long as the amount of the ferrate does not interfere with the resulting mechanical integrity of the fiber. The concentration of the ferrate can be in the range of up to about 25% v/v depending on the application. For example, in a pad or brush made of fibers containing ferrate, a higher concentration of the ferrate is needed to ensure immediate release of the free ferrate to react with water or other contaminants/molds etc.

The concentration of the ferrate in a sample can be determined by UV/vis spectrophotometry by comparing the concentrations of ferrate(VI) determined by dissolving in an aqueous solution of 32% NaOH and measuring the absorbance readings at 505 and 785 nm. For the measurement to be qualitatively and quantitatively correct, the concentrations derived from each absorbance reading should be the same within 2-10%. Also, the presence of colloids or particulates is indicated that scatter light causing a false high in the concentration measurement. In this event, further purification of the analytical sample is required, by centrifugation or filtration, to remove these particulates and/or colloids and the absorbance is then re-read at the two diagnostic wavelengths. In addition, for accurate measurement, care should be taken to avoid conditions that would change the oxidation state of iron. The NaOH solution should be free of reducing agents. If necessary, where a ferrate(VI) containing composition is strongly hydrophobic, the hydrophobic matrix may be removed (such as with toluene or phase transfer catalyst, i.e. quaternary ammonium ion, a blend of quaternary ammonium ions, phosphonium ion, a blend of phosphonium ions), for example by water washing the ferrate(VI) ion from the hydrophobic phase, just prior to dissolving the ferrate(VI) in 32% NaOH.

Unexpectedly, unlike all peroxide materials, we found potassium ferrate(VI) to be thermally stable as defined by Army Regulation (AR) 70-38 test conditions. Therefore potassium ferrate(VI) was determined to be stable to storage for long periods, at least 98 days at 71° C., and at least 82 days under conditions of cycling daily from 23° C. to 71° C. and back again. This result is true regardless of whether the potassium ferrate(VI) is pure (80-100% analytical grade) or of only moderate purity (50-80% technical grade). In these tests, losses with time varied slightly with test vials ranging from <1% to 10% (±3%) loss respectively after the test periods given. From these results, it can be estimated that the potassium ferrate(VI) will likely be sufficiently stable for several years of storage.

The ferrate fiber of the present invention can disperse the ferrate formulation mentioned above into one or more suitable polymers to produce the ferrate fiber. Broadly, a formulation of the ferrate is mixed/blended with at least one nonaqueous polymer, and then the mixture can be dispersed and cooled to solidify into the fiber of the present invention. The nonaqueous polymer is defined above. A conventional extrusion method can be used to co-extrude/disperse the ferrate/polymer mixture. Preferably, the polymer is in a liquid form before blending with the ferrate formulation. The polymer can be melted into a liquid polymer before blending. The ferrate is highly oxidative so it is normally not placed in contact with any high temperature organic polymer, nor is it co-extruded/dispersed with other polymers under a relatively high temperature using a conventional extrusion method. Such blending and co-extrusion might decompose the ferrate prematurely. Surprisingly, sodium ferrate(VI) is stable up to about 270° C. Most suitable polymers can melt at a much lower temperature. Therefore, the ferrate(VI) can be blended and then co-extruded/dispersed with one or more melted liquid polymers without causing the ferrate to decompose prematurely even using a relatively vigorous conventional extrusion method.

Preferably, the fiber of the present invention can be produced using electric field effect technology. Electric Field Effect Technology (called “EFET” herein thereafter) includes electrospraying, electrohydrodynamic spraying (EHD), electric field spraying, electro-spinning, spray technology as exemplified by patents such as U.S. Pat. No. 6,252,129 to Coffee, U.S. Pat. Pub. No. 2009/0104269 to Graham et al., U.S. Pat. Pub. No. 2008/0259519 to Cowan et al., U.S. Pat. Pub. No. 2006/0194699 to Moucharafieh et al. (the contents of these patent and published patent application are hereby incorporated by their entirety), and the like. Further, “EFET” can be used interchangeably with “EHD.”

Electrospraying as used herein means conventional spraying with an electrical component added. Electrohydrodynamic spraying (EHD) is a process of spraying a bulk formulation by using electric forces. An electrical charge is applied to the fluid so that as it exits from the spray site, it forms a cone-jet geometry. The jet may break up into aerosol droplets or particles or solidify to form a fiber. Electric field spraying is a process of electrohydrodynamic spraying whereby the formed jet subsequently breaks up into particles or fibrils (truncated fibers). Electro-spinning is a process of electrohydrodynamic spraying whereby the formed jet solidifies to a fiber, which may be utilized in this form or collectively combined to form a fibrous mat. EFET uses these technologies to effectively produce an electric field induced cone-jet or Taylor cone for producing the ferrate embedded fibers (the ferrate fibers of the present invention).

In other words, EFET embodies the process of utilizing an electric field to charge and subsequently extrude aerosol particles of microstructures, such as fibers, films or nano/microparticles etc., from a bulk liquid formulation. In the present invention, EFET is used to produce fibers of ferrate compounds. The size of the microstructures can be adjusted from fractions of a micron to hundreds of microns, depending on the specific application. Because the microstructures from EFET are electrically charged when they are formed, additional features may be leveraged, such as directivity of the microstructure through electrical means, and interactions among the generated microstructures to cause secondary formations, such as fiber mats. Distinctive advantages of EFET include its flexibility to produce a variety of structures, consistency of performance, and gentle handling of delicate materials, such as the highly oxidative ferrate(VI) compounds.

In addition, the EFET method for producing the ferrate fiber is preferred because the fiber size can be generally produced within a tight distribution range. The fiber is also produced such that the solvent for the ferrate can be evaporated or flashed off quickly. Further, the resulting ferrate fiber can also be coated or encapsulated in one continuous step using two or more nozzles via EFET.

The EFET technology can be used to encapsulate the ferrate crystals. Encapsulation as used herein means that the ferrate crystal is at least partially or completely encapsulated by an encapsulant. An at least partially encapsulated ferrate in some embodiments typically includes a ferrate crystal embedded in or adhered to a carrier material, such as the nonaqueous polymer or other suitable excipients or solvents.

When utilizing EFET to produce the ferrate fiber of the present invention, voltages in the range from about 2 kV to about 25 kV are contemplated with the different embodiments of the invention. For some embodiments, a nonaqueous liquid formulation of the ferrate is co-sprayed with an encapsulant liquid formulation typically containing at least one suitable nonaqueous polymer using various nozzle configurations. Graham et al. in U.S. Pat. Pub. No. 2009/0104269 disclose three types of nozzles: concentric nozzle, Siamese nozzle, and Gemini nozzle (the entire content of the published patent application by Graham et al. are incorporated herein). Piatt et al. in a PCT patent application publication no. WO2004/062812 disclose various spray head useful for the EFET application.

The ferrate crystal, thus associated with a fiber in solid, semi-solid or gel form, will have different properties, such as quick dissolve, slow dissolve, controlled release etc. Quick dissolve format fibers have been efficiently created by the inventors using an EFET fiber spraying process previously disclosed in patents such as U.S. Pat. No. 6,252,129. However, due to its high oxidative capability, prior to the present invention, the ferrate has not been contemplated to be mixed with organic polymers to produce the ferrate fibers using EFET. In the present invention, EFET is used to incorporate the ferrate with organic polymers into the fiber in such a way that the release of the ferrate can be accomplished in a controlled fashion.

In other words, the ferrate fiber of the present invention can be created using the EFET process described in U.S. Pat. No. 6,252,129 to Coffee, U.S. Pat. Pub. No. 2009/0104269 to Graham et al., U.S. Pat. Pub. No. 2008/0259519 to Cowan et al., U.S. Pat. Pub. No. 2006/0194699 to Moucharafieh et al., from the nozzle configuration of U.S. Pat. Pub. No. 2009/0104269 and/or from the spray head of a PCT patent application publication no. WO2004/062812. Alternatively, two separate ferrate fibers may be produced from separate nozzles. Further, multiple fibers or fibrils of the ferrate may be used, in which one ferrate fiber is produced using an electrohydrodynamic spraying method (or other EFET methods), and the other ferrate fiber can be produced from an electro-spinning method, or other EFET methods, or from other conventional co-extrusion methods.

In the present invention, three main factors of EFET need to evaluated and developed in producing the ferrate fibers of the present invention: (1) device and formulation development; (2) the establishment of the electric field by the device with its environment; and (3) the formation of the microstructures.

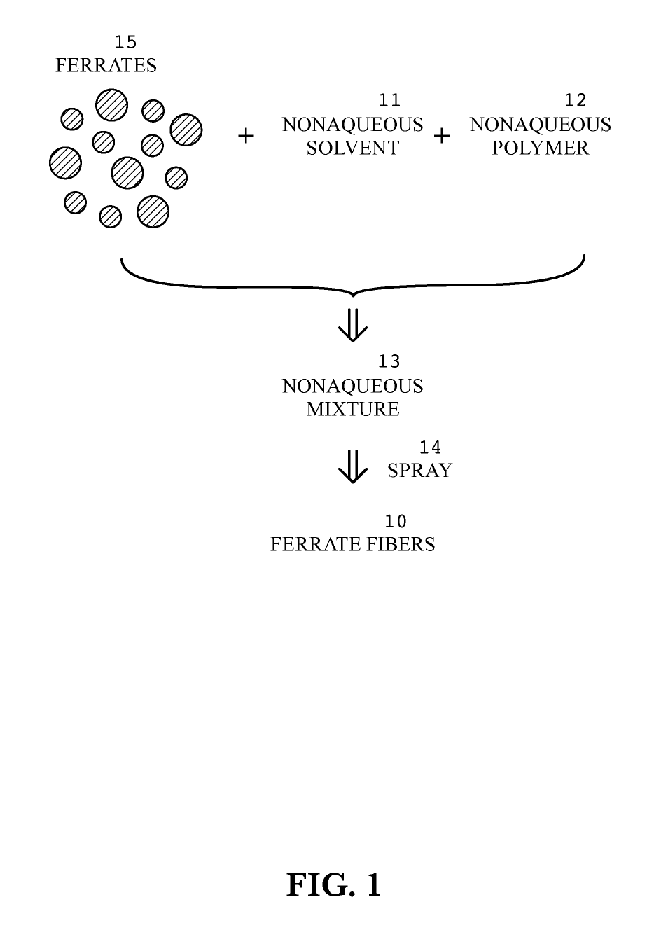

According to FIG. 1, the ferrates 15 (or ferrate compounds) need to be combined with at least one suitable nonaqueous solvent 11 and with at least one nonaqueous polymer 12. The preferred solvent 11 should provide adequate resistance to oxidation by the ferrate, be capable of dissolving the nonaqueous polymer, and possess sufficient volatility so that the solvent can be substantially evaporated during the ferrate fiber production process. Unlimited examples of the preferred solvents are described above. If the preferred solvent is adequate or strongly resistant to oxidation by the ferrate, the ferrate can be dissolved in the solvent. On the other hand, if the solvent is only weakly or not resistant to oxidation to the ferrate, the ferrate should be substantially insoluble in the preferred solvent. Otherwise, the ferrate can be encapsulated or put into a solid solution so that the encapsulated ferrate or ferrate solid solution is substantially insoluble in the solvent. Further, the solvent should have such volatility that a majority of the solvent is evaporated at the time that the ferrate fiber is produced, but a sufficient amount of the solvent is left to ensure the resulting ferrate fiber is not too dry or too brittle.

The nonaqueous polymer 12 is used to provide sufficient viscosity to the formulation to enable the production of the ferrate fiber of the present invention. As defined above, the nonaqueous polymer 12 preferably contains 3 wt % or less water.

Preferably, the nonaqueous polymer 12 is strongly resistant to oxidation by the ferrate, in which case, the ferrate can be soluble in the polymer 12. In case that the nonaqueous polymer 12 is somewhat or weakly or not resistant to oxidation by the ferrate, then the ferrate should be substantially insoluble in the polymer 12; or the ferrate can be encapsulated or put into a solid solution so that the encapsulated ferrate or the ferrate solid solution is substantially insoluble in the polymer 12. Unlimited examples of the preferred nonaqueous polymer 12 are given in other parts of this application.

The resulting nonaqueous mixture 13 (FIG. 1), then, is composed of ferrates 15 suspended or dissolved in a nonaqueous solution in which the polymer 12 is dissolved in the nonaqueous solvent 11. Such a mixture 13 is sprayed 14 by electrohydrodynamics (EHD) to produce ferrate fibers 10 of the present invention. Typically, the resulting ferrate fibers have a diameter of less than about 1000 nm.

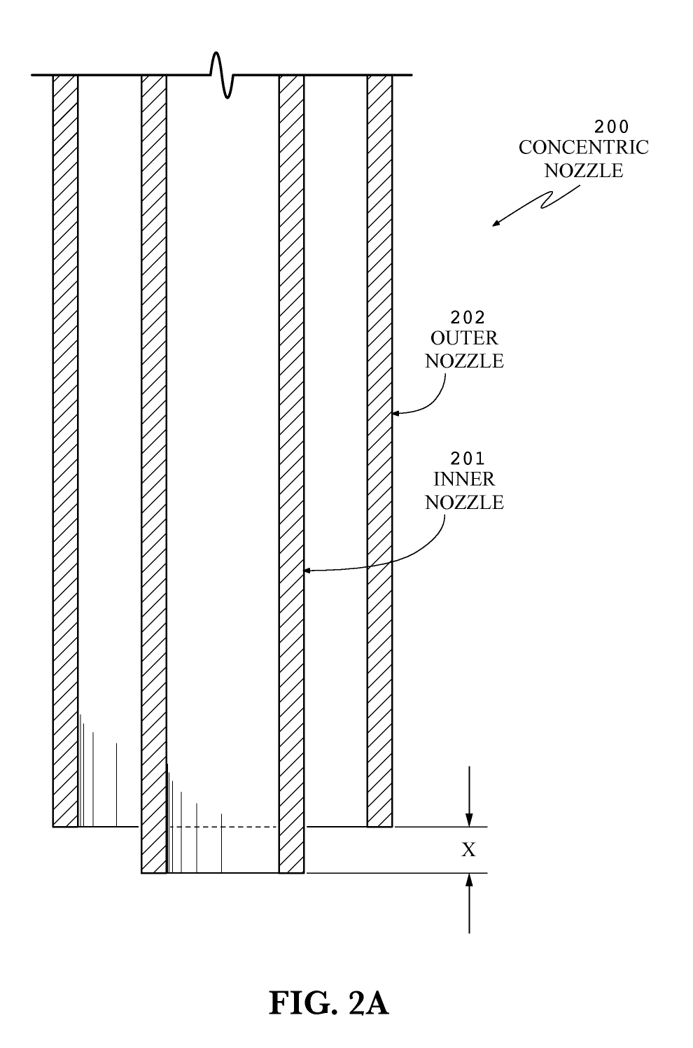

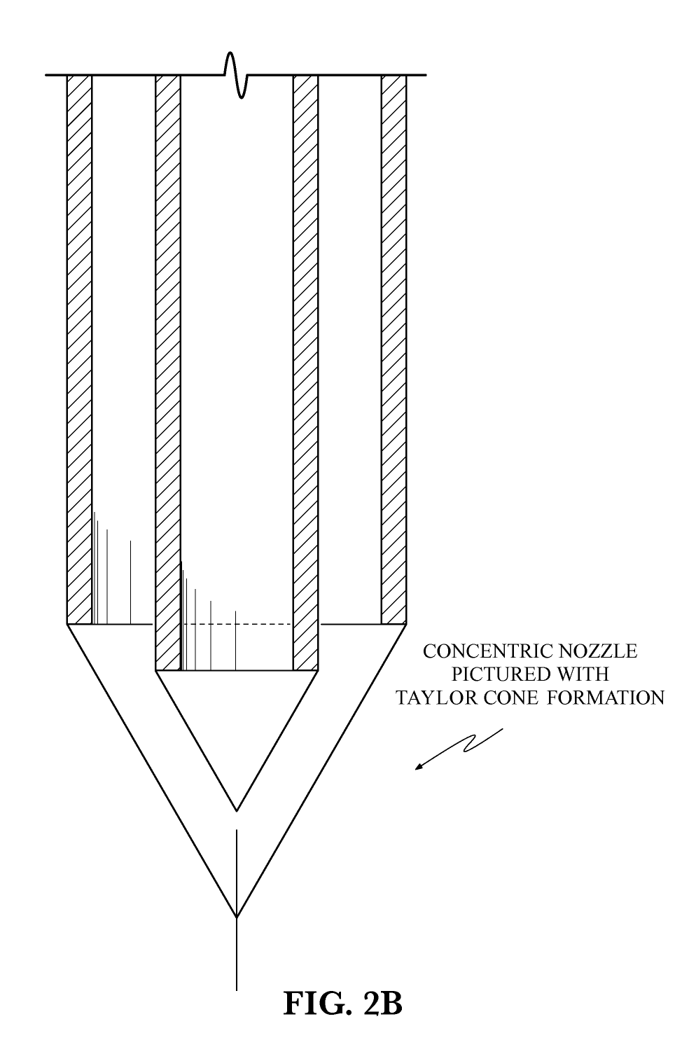

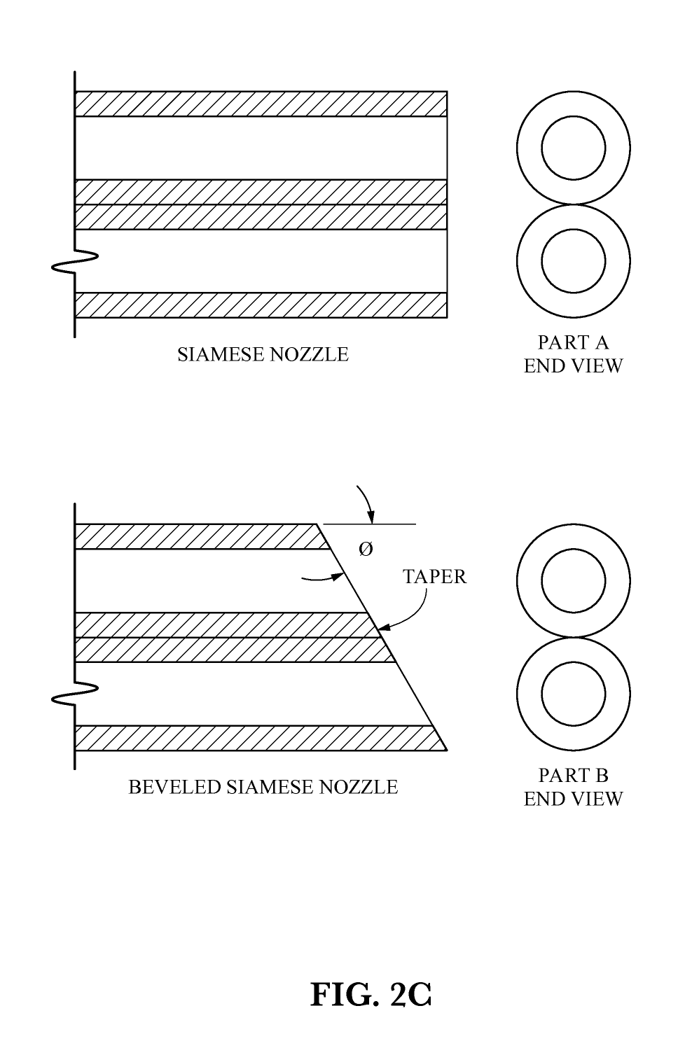

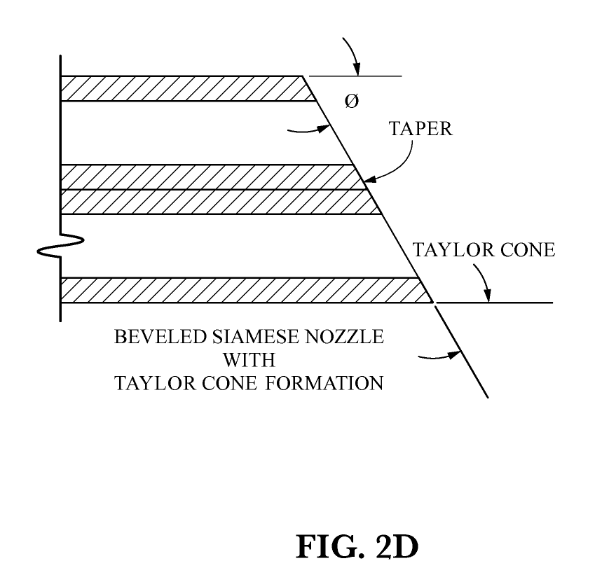

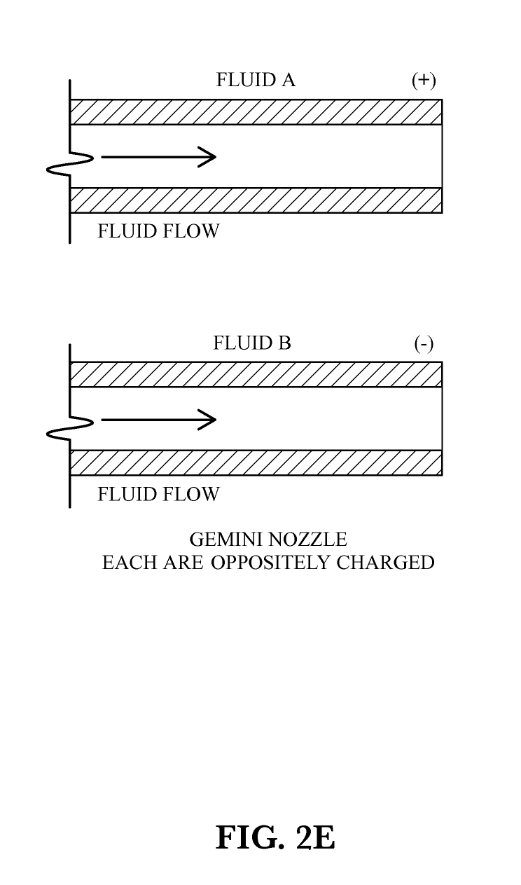

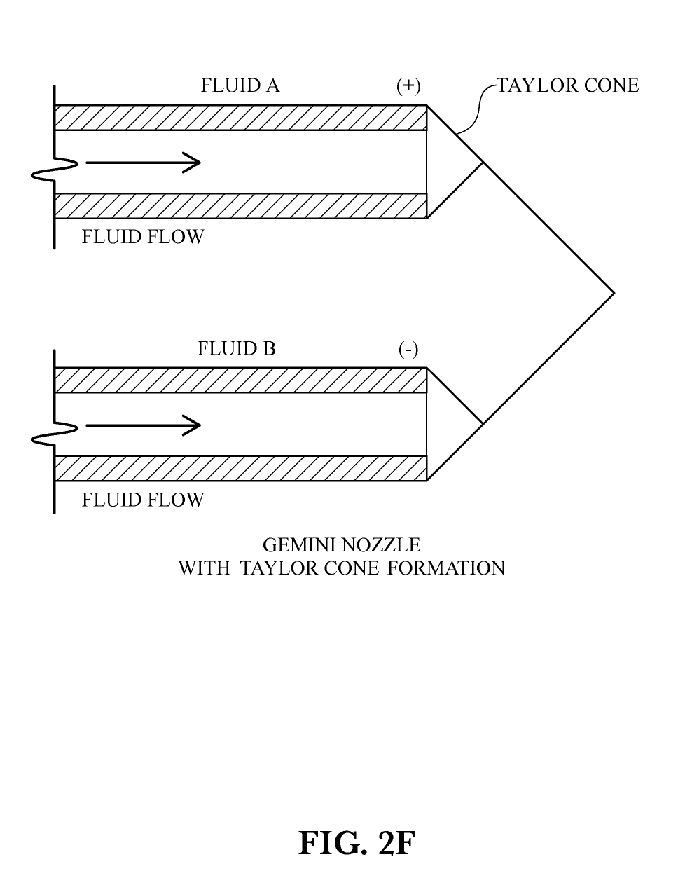

In a typical EHD spray nozzle (see FIGS. 2A-2F), the fluid to be aerosolized flows over a region of high electric field strength. When it does so, it receives a net electric charge that tends to stay on the surface of the fluid. Hence, as the fluid exits the spray site, the repelling force of the surface charge balanced against the surface tension of the material, and a cone is formed (known as a Taylor cone). The tip of the cone has the greatest concentration of charge, and at this point, the electrical force overcomes the surface tension, generating a thin jet of fluid. The jet forms into fibers, continuous or discontinuous, which flow along the electric field lines. In the present invention, it is advantageous to maintain the imparted charge on the fibers. When the electrically charged fiber is directed toward and strikes a target surfaces, it forms a non-woven mat of more or less randomly oriented (without other electrical or mechanical influences) fibers having consistent size and porosity. These mats can be created in situ or in a full scale manufacturing setting.

Referring now to FIGS. 2A through 2F, various nozzle configurations can be used to spray or co-spray the mixture 13 to produce the ferrate fiber 10 using EFET technologies. Three nozzle configurations are Concentric nozzle, Siamese nozzle, and Gemini nozzle. Other configurations for spraying one liquid, two liquids, one liquid and one solid, one liquid and one aerosol et c. are also feasible via EFET. The nozzle configurations are not limited to one or two nozzles and can be extended to have an array of nozzles that perform a similar task.

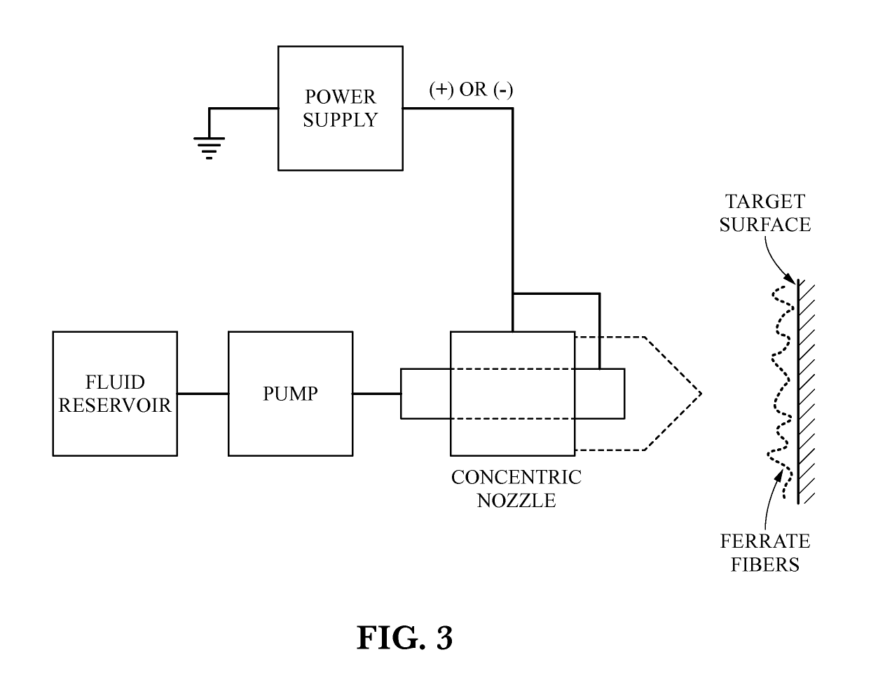

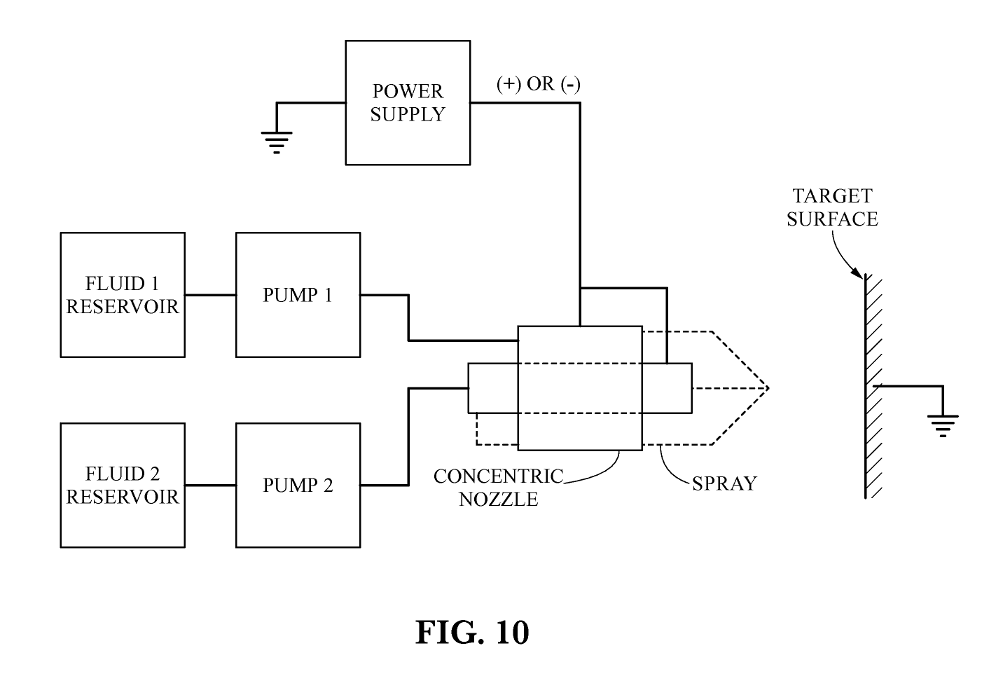

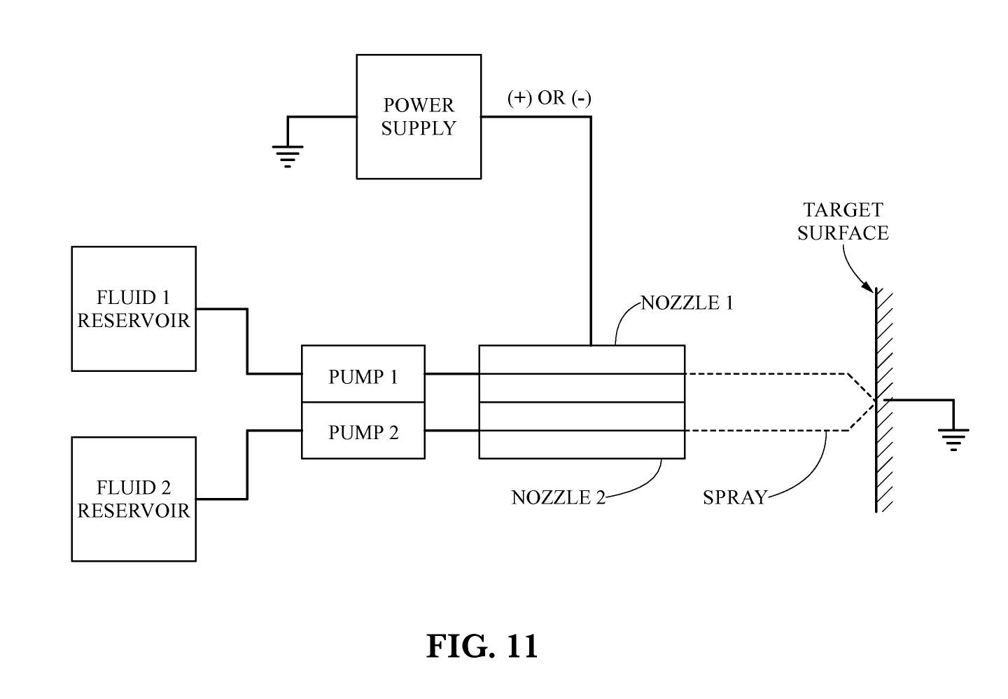

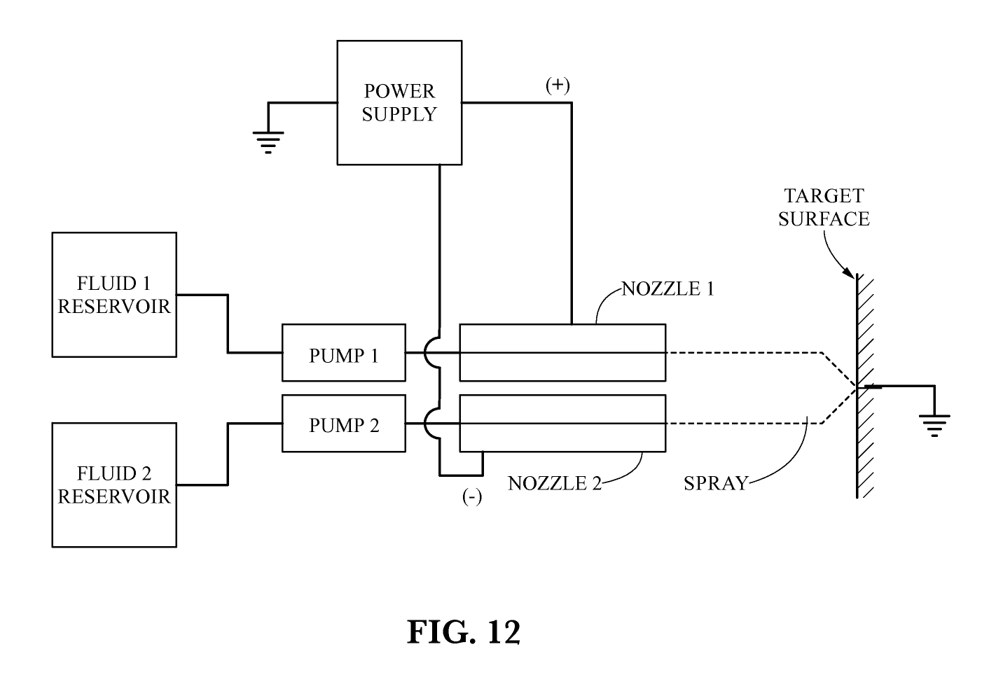

FIGS. 2A-2B illustrate a typical Concentric nozzle 200 used in the example. This design is an inner nozzle 201 inside an outer nozzle 202. Each of the nozzles is a tube, and the sizes of the tubes were calculated to make sure that the inner tube fit into the outer tube with some clearance space. That clearance space was the fluid path of the outer nozzle, while the inner diameter of the inner tube was the fluid path of the inner nozzle. If one pumping mechanism with one fluid reservoir as shown by FIG. 3 is used, then the inner and outer nozzles should be sized to give approximately the same fluid velocity exiting the tubes. However, different fluid velocities can be used for inner and outer nozzles depending on the particle sizes and fluid properties. Further, different fluids can be pumped through one or more nozzles with different flow rate using multiple pumps (see FIGS. 10-12).

In the example, the outer nozzle had an internal diameter of about 0.050″ and an outer diameter of about 0.058″. The inner nozzle had an internal diameter of about 0.020″ and an outer diameter of about 0.028″. The nozzles were charged at the same polarity, positive in this case. Fibers embedded with ferrate nanoparticles using EFET were produced at a laboratory scale level. This was accomplished by spraying the suspension mixture 13 through both nozzles. The resulting sprays were collected on glass slides, tested for the presence of the active ferrate compounds, and then viewed under a microscope.

Alternatively, the polymer can be dissolved in the solvent producing a polymer solution, while the ferrate can be suspended in the solvent, which can be same or different from the solvent used to dissolve the polymer, to produce a ferrate suspension. The polymer solution can be sprayed through the outer nozzle 202, and the ferrate suspension can sprayed through the inner nozzle 201. In FIG. 2A, the inner nozzle 201 may extend beyond the outer nozzle 202 by a distance X. A Taylor cone is formed as shown in FIG. 2B.

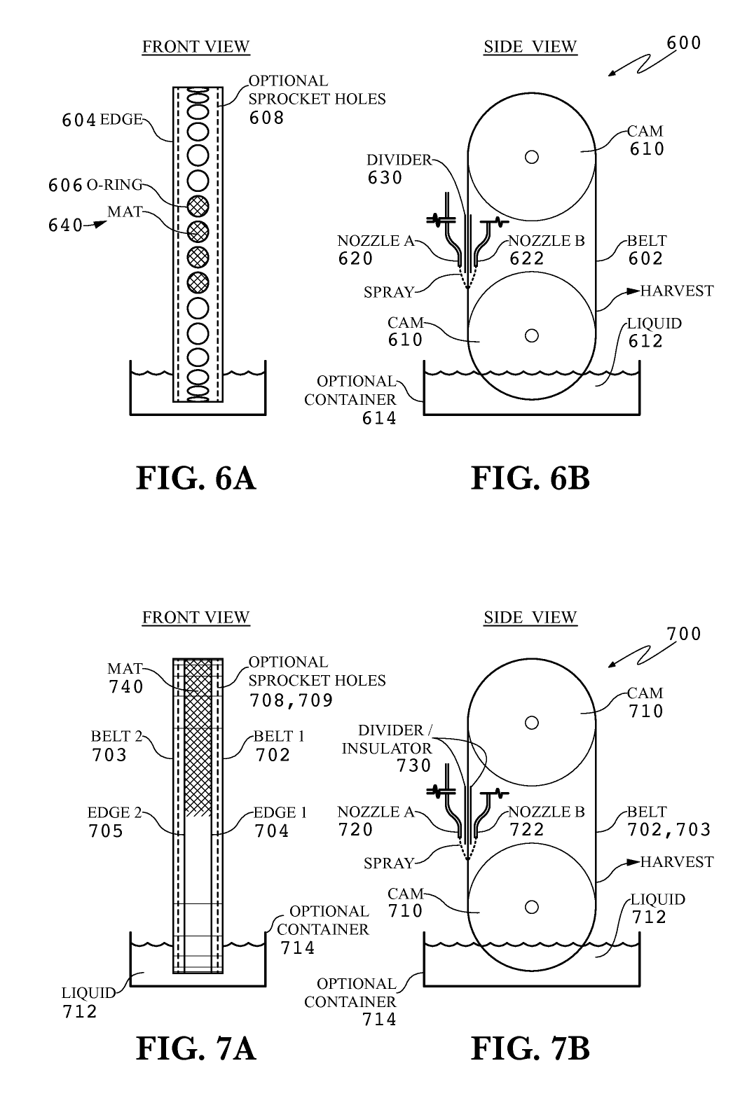

In some other embodiments, the ferrate polymer mixer is pumped through the inner nozzle, while a coating solution is pumped through the outer nozzle. The final product is a coated ferrate fiber. Or in some further embodiments (FIGS. 6A-7B), the ferrate fiber produced above can be passed through an optional container containing a coating liquid, which applies a layer of coating to the ferrate fiber.

FIGS. 2C-2D illustrate a typical Siamese nozzle. This nozzle configuration includes twin nozzles attached adjacent to one another with the ends normal to the same plane. The nozzles are probe stainless steel point hypodermic needles attached together with a small diameter (about 0.010″) stainless steel wire. The nozzles are preferably charged with same potential, positive typically. One fluid or different fluids can be pumped through the nozzles with a single pumping mechanism (FIG. 3) to produce the same flow rate in each nozzle. Multiple pumping mechanisms as shown by FIGS. 10-12 can be used if different flow rates per nozzle are required.

In some embodiments, in order to provide smooth fluid transition at the end of the nozzle where the Taylor cone would form, the ends of the nozzles are beveled or mitered to an angle as shown in FIGS. 2C-2D. The bevel is preferably created by surface treatment of the nozzle at an approximate 45 degree angle to the surface of fine grit sand paper. FIG. 2C part B shows a Siamese nozzle that has a tapered surface of angle . This allows the fluids to mix as they flow out of the ends, and then aerosolize at the outermost tip. The beveled edges are advantageous with this set of fluids to create a Taylor cone that helped in creating the ferrate fiber end product. The spray reaches the target surfaces, such as glass slides, to produce the ferrate fibers.

FIGS. 2E and 2F illustrate a typical Gemini nozzle. The concept of this nozzle is to use two different nozzles separated from each other, one nozzle is positively charged, while the other is negatively charged. When opposing charges are used the resulting aerosol or spray would have enhanced mixing or capture of the formulation after atomization, thus reducing problems related to formulation, solvent, and active ingredient compatibility.

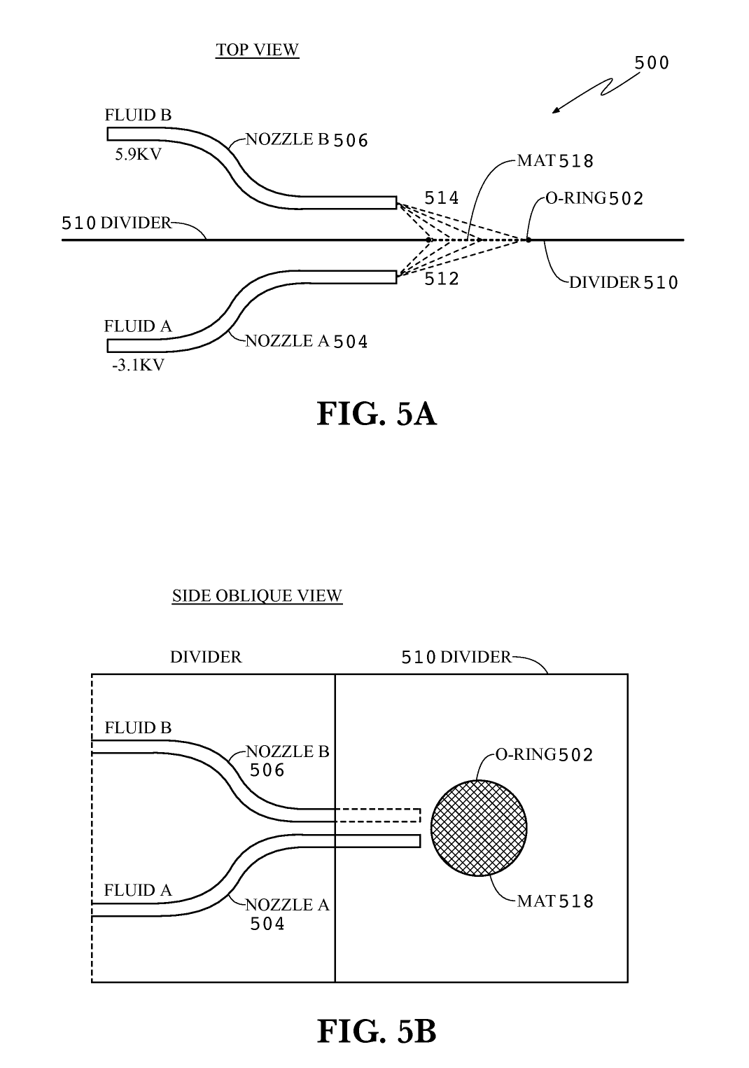

The fibers formed by the present invention may be collected with an apparatus as described to FIGS. 5A through 7B. FIGS. 5A and 5B illustrate a discontinuous method using apparatus 500 with one circular edge (e.g. O-ring) 502. Fluids A and B are co-sprayed form nozzle A 504 and nozzle B 506 respectively. An optional divider 510 may be used to separate sprays 512 and 514 until they reach the desired location for combining, where a fiber mat 518 is formed in the space within the O-ring 502.

FIGS. 6A and 6B illustrate an apparatus 600 having a continuous belt 602 with circular edges 604 typically formed by an 0-ring 606. The edges may also be oval square, or rectangular, with the circular edges or regions being shown in the figures. The belt 602 may have sprocket holes 608 for advancing and keeping the belt 602 aligned and typically runs on a cam 610. When an O-ring is filled with fiber, the continuous band moves so that the next O-ring is positioned to accept the fibers from the spray nozzles. The fibers can then be harvested from the O-ring. The belt 602 can optionally move through a bath of nonaqueous liquid 612 for coating the collected fiber material. The liquid may be held in an optional container 614. Nozzles A and/or B, 620 and 622, respectively, are used to spray the liquids. An optional divider 630 may be used. A ferrate fiber matt 640 typically forms at the edge 604.

FIGS. 7A and 7B illustrate an apparatus having a dual belt 702 and 73 and double set of edges 704, 705 within a set of a continuous band. The continuous bands may include a movable set of plurality of circular edges, or a movable double continuous belt having two edges (Edge 1, Edge 2) with a central opening. The belts 702, 703 may have sprocket holes 708, 709 for advancing and keeping the belts 702, 703 aligned and typically run on a cam 710. When one area of the belts is filled with fiber, the continuous band moves so that the next open area is positioned to accept the fibers from the spray nozzles. The belts 702, 703 may optionally move through a bath of nonaqueous liquid 712 for coating the collected ferrate fiber material. The liquid may be held in an optional container 714. Nozzles A and/or B, 720 and 722 respectively, are used to spray the liquids. An optional divider 730 may be used. A ferrate fiber mat 740 typically forms between the edges.

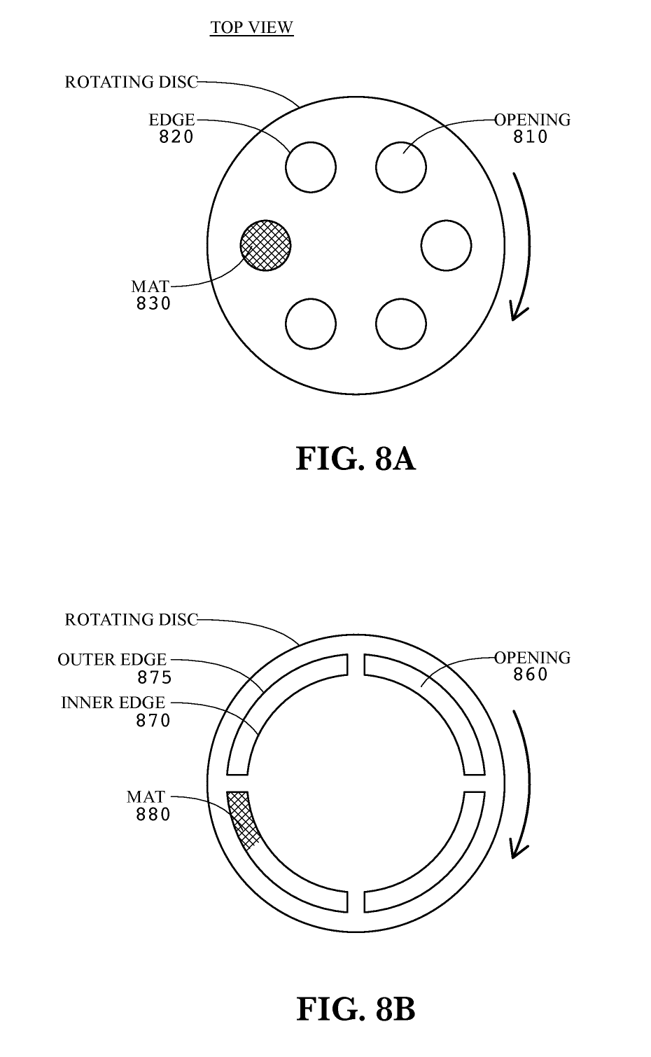

FIGS. 8A and 8B illustrate rotating discs, respectively that can be used in the formation and/or collection of the ferrate fiber mats. FIG. 8A illustrates circular openings 810 that provide circular edge 820 for mat 830 formation. FIG. 8B illustrates semicircular openings 860 that are arc shaped. Mats 880 are built up between the inner edge 870 and the outer edge 875 as shown. Nozzles and belt configuration illustrated in FIGS. 6A-7B and 9 may be used with the discs of FIGS. 8A and 8B.

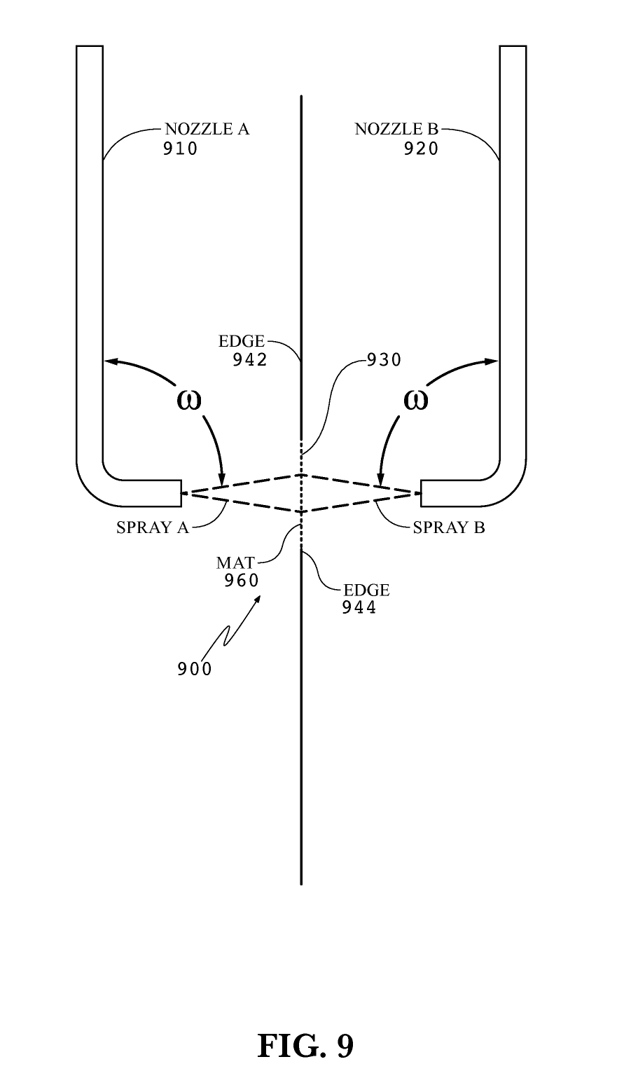

FIG. 9 illustrates another embodiment of the invention that provides for an apparatus 900 having nozzles A 910 and B 920 that are configured so as to spray to each other. A collection area 930 between two edges 942, 944 may be used to build a mat 960 between two edges as shown. In some embodiments there may be no edges, and the nozzles produce fibers or fibril. Regarding the spray head, the angle ω is typically about 90°. However, for some embodiments, angle ω may be anywhere between about 30° and about 180°.

In some other embodiments of the invention, the apparatus provides for a partial vacuum in the area of the spray tip or tips. The sprayed materials are sprayed into a partial vacuum. The partial vacuum provides for enhanced solvent flashoff. This can enhance speed of the process, enhance flashoff of the solvents or co-solvents that have higher boiling points, or have larger surfaces, or otherwise are more difficult to remove. The partial vacuum may also enhance removal of certain excipients listed herein that are not needed or desired on or within the formed ferrate fibers.

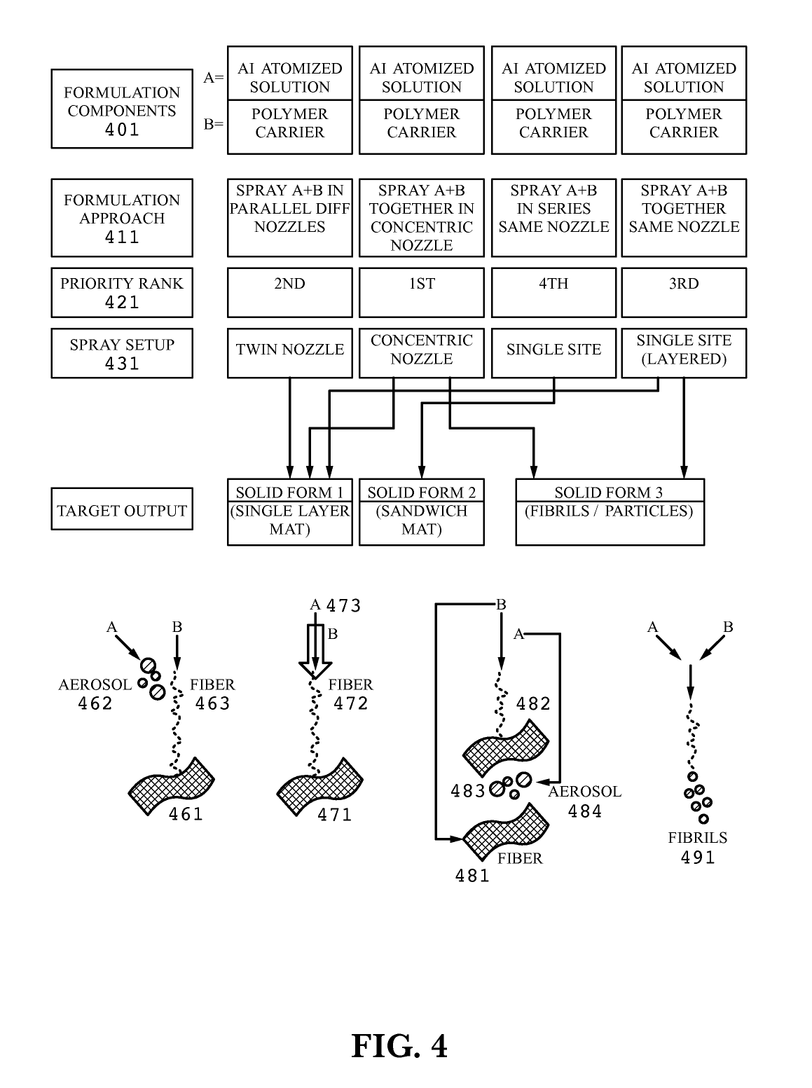

Several embodiments of the present invention are illustrated in FIG. 4. Formulation Components 401, having parts A and B, are listed to the right; Formulation

Approaches 411 are listed to the right; Priority Ranking 421 in terms of presently preferred approaches are listed to the right; Spray Setup Configurations 431 are listed to the right; and Target Outputs are also listed to the right.

The first illustration at the bottom of the page is a single layer mat 461 that incorporates discrete aerosol ferrate particles 462 into a fiber 463; the second illustration is for another single layer mat 471 that incorporates fibers 472 with ferrate particles 473 into a mat 471; the third illustration shows a sandwich mat 481 made from an upper layer of fiber 482 from component B, a lower layer of fiber 484 from component B and middle layer of particles 483; and the fourth illustrations shows fibrils 491 made from components A and B.

The resulting ferrate fiber can be used for purposes of whitening, cleaning, disinfection, deodorizing, oxidizing/removing a variety of organic and inorganic compounds/contaminants from aqueous and non-aqueous (air) media. To accomplish these purposes, the fiber can be made into pads, brushes, or filters as mentioned above.

The present invention is further illustrated by the following example which is illustrative of some embodiments of the invention and is not intended to limit the scope of the invention in any way:

### Example

This example evaluates the feasibility of incorporating potassium ferrate(VI) crystals into fibers using an EFET technology (device plus process). The EFET device and process for the example are illustrated in FIGS. 3, including one fluid reservoir, one Concentric nozzle, and one pump. The Concentric nozzle was as illustrated in FIGS. 2A-2B.

The formulation components used in this example were as illustrated in FIG. 1. The ferrates were sodium ferrate crystals produced in our own laboratories. A nonaqueous polymer, polyvinylpyrrolidone (PVP), was first dissolved in one or several preferred nonaqueous solvents. The preferred solvents include menthol, ethanol, ether, toluene, isopropyl alcohol, acetonitrile (CH₃CN), tetrahydrofuran (THF), dimethylsulfoxide (DMSO), N-methylpyrrolidone (NMP), and dichloromethane (CH₂Cl₂). The solvents used were acetonitrile, toluene, and dichloromethane. Some of the solvents were treated to remove excess water so that they preferably contained no more than 3 wt % water. The sodium ferrate crystals were then mixed into the polymer solution to create a nonaqueous ferrate suspension, where the ferrate was substantially insoluble in the solvents used.

The ferrate suspension then was pumped through the Concentric nozzle of FIGS. 2A-2B. The outer nozzle had an internal diameter of about 0.050″ and an outer diameter of about 0.058″. The inner nozzle had an internal diameter of about 0.020″ and an outer diameter of about 0.028″. The nozzles were charged at the same polarity, positive in this case. The ferrate mixture 13 was sprayed through both inner and outer nozzles. The resulting sprays were collected on glass slides, one of which was covered with phosphate buffer, while the other one is covered with nitric acid.

After spraying the glass slides with the ferrate mixture via EFET, the phosphate buffer slide turned purple, while the nitric acid slide turned pale yellow or almost colorless. The color change demonstrated that the ferrate in the fiber was still active, indicating that the process of incorporating the ferrates into the fiber via EFET did not inactivate the ferrate compound.

The ferrate fibers collected on the glass slides were then viewed under a microscope. Mat shaped fibers were observed. Further, the crystals of ferrate(VI) appeared to be mostly embedded inside the fiber with some ferrate(VI) crystals extending out of the fiber. The exposed portions of the ferrate crystals were observed to be rusty looking, suggesting that the exposed ferrate crystals were decomposed while the embedded ferrate crystals were protected from premature decomposition.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

1. A fiber containing ferrate, comprising
a. one or more ferrate compounds, wherein the ferrate compound is selected from a group consisting of a metal ferrate(V) compound, metal ferrate(VI) compound, and a mixture thereof; and
b. one or more nonaqueous polymers.

2. The fiber of claim 1, wherein the metal ion of the ferrate compound is selected from a group consisting of an alkali metal, alkaline earth metal, an oxidation resistant transition metal from Groups IIB & IIIB, a Group (III)A metal, lanthanide metal, and a combination thereof.

3. The fiber of claim 1, wherein the metal ion of the ferrate compound is selected from non-oxidizable oxidation states of Li, Na, K, Rb, Cs, Mg, Ca, Sr, Ba, Sc, Y, La, other lanthanide, Zn, Cd, Al, Ga, In, Tl, Pb(II), Bi(II), or mixtures thereof.

4. The fiber of claim 1, wherein the ferrate compound is encapsulated.

5. The fiber of claim 1, wherein the ferrate ion is embedded in a solid solution with one or more compatible ions.

6. The fiber of claim 5, wherein the compatible ion comprises a sulfate ion, a chromate ion, a silicate ion, an aluminate ion, an orthophosphate ion, a borate ion, a carbonate ion, a titanate ion, a zirconate ion, a manganate ion, a molybdate ion, or a mixture thereof.

7. The fiber of claim 1, wherein the nonaqueous polymer comprises epoxy resin, alkyd, polyester, polyurethane, polyolefin, polyamide, polysulfide, polythiolether, phenolic polyether, polyurethane, polyvinyl, rosin polyesters, silicones, siloxanes, perfluorinated resin, other fluorinated resin, polytetrafluoroethylene, polyvinylidene difluoride, nylon and other polyamide, a copolymer thereof, or a mixture thereof.

8. The fiber of claim 1, further comprising one or more nonaqueous solvents.

9. The fiber of claim 8, wherein the nonaqueous solvent comprises xylene, toluene, acetonitrile, dichloromethane, tetrahydrofuran, dimethylsulfonide, petroleum distillate, ketone, N-methyl pyrrolidone, triethanolamine, 2-ethoxyethanol, other nonaqueous solvent, alcohol, or a mixture thereof.

10. The fiber of claim 1, further comprising one or more excipients, preferably nonaqueous excipients.

11. The fiber of claim 10, wherein the excipient comprises an amphiphilic material, a surfactant, a pH buffer, an alcohol, an encapsulation agent, a phase transfer catalyst, a wetting agent, a dispersant, a binder, a plasticizer, a gelling material, a caustic agent, a thickener, an accelerant, an emulsifier, an optional de-colorant, a humectants, an optional colorant, an optional antifungal, an optional antibacterial, or a mixture thereof.

12. The fiber of claim 11, wherein the amphiphilic material comprises cationic surfactants, anionic surfactants, non-ionic surfactants, quaternary surfactants, amphoteric surfactants, zwitterionic surfactants, and combinations thereof.

13. The fiber of claim 10, wherein the excipients comprises mono-phosphate, poly-phosphate, bicarbonate salt, calcium carbonate, sulfate salt, orthophosphate ester, orthophosphate salt, tetraorganoammonium ion, pyrophosphate salt, titanium dioxide, clay silicate, aluminum silicate, aluminate, aluminosilicate, talc, mica, silica, silicate, magnesium silicate, zinc oxide, barite sulfate, barium sulfate, or a mixture thereof.

14. The fiber of claim 1, wherein the ferrate compound is present in a concentration that does not interfere with the mechanical integrity of the fiber.

15. The fiber of claim 1, wherein the ferrate compound is present in a concentration of up to about 25 v/v %.

16. A method of making a fiber containing ferrate, comprising
a. providing a nonaqueous formulation of one or more ferrate compounds, wherein the ferrate compound is selected from a group consisting of metal ferrate(V) compounds, metal ferrate(VI) compounds, or mixtures thereof;
b. providing one or more nonaqueous polymers; and
c. dispersing the ferrate formulation of step (a) in the nonaqueous polymer to produce the fiber.

17. The method of claim 16, wherein the step (d) is accomplished by means of electric field effect technology.

18. The method of claim 16, wherein a metal of the ferrate compound is selected from comprises Li, Na, K, Rb, Cs, Mg, Ca, Sr, Ba, Sc, Y, La, other lanthanide, Zn, Cd, Al, Ga, In, Tl, Pb, Bi, or a mixture thereof

19. The method of claim 16, wherein the ferrate formulation of step (a) comprises one or more nonaqueous solvents, and/or one or more excipients.

20. The method of claim 19, wherein the excipient comprises an amphiphilic material, a surfactant, a pH buffer, an alcohol, an encapsulation agent, a phase transfer catalyst, a wetting agent, a dispersant, a binder, a plasticizer, a gelling material, a caustic agent, a thickener, an accelerant, an emulsifier, an optional de-colorant, a humectants, an optional colorant, an optional antifungal, an optional antibacterial, or a mixture thereof.

21. The method of claim 16, wherein the ferrate compound formulation comprises one or more encapsulated ferrate compounds.

22. The method of claim 16, wherein a ferrate ion of the ferrate compound is embedded in a solid solution with one or more compatible ions.

23. The method of claim 22, wherein the compatible ion comprises a sulfate ion, a chromate ion, a silicate ion, an aluminate ion, an orthophosphate ion, a borate ion, a carbonate ion, a titanate ion, a zirconate ion, a manganate ion, a molybdate ion, or a mixture thereof.

24. The method of claim 16, wherein the nonaqueous polymer comprises epoxy resin, alkyd, polyester, polyurethane, polyolefin, polyamide, polysulfide, polythiolether, phenolic polyether, polyurethane, polyvinyl, rosin polyesters, silicones, siloxanes, perfluorinated resin, other fluorinated resin, polytetrafluoroethylene, polyvinylidene difluoride, nylon and other polyamide, a copolymer thereof, or a mixture thereof.

25. A method of cleaning and/or disinfecting using a pad or a brush composed of fibers containing ferrate, wherein the fiber comprises
a. one or more ferrate compounds, wherein the ferrate compound is selected from a group consisting of metal ferrate(V) compounds, metal ferrate(VI) compounds, or mixtures thereof; and
b. one or more nonaqueous polymers.

26. A method of treating air or water using fibers containing ferrate, wherein the fiber comprises
a. one or more ferrate compounds, wherein the ferrate compound is selected from a group consisting of metal ferrate(V) compounds, metal ferrate(VI) compounds, or mixtures thereof; and
b. one or more nonaqueous polymers.

27. A pad or a brush suitable for cleaning or disinfection, comprising fibers containing ferrate, wherein the fiber comprises
a. one or more ferrate compounds, wherein the ferrate compound is selected from a group consisting of metal ferrate(V) compounds, metal ferrate(VI) compounds, or mixtures thereof; and
b. one or more nonaqueous polymers.

28. A filter suitable for air or water treatment, comprising fibers containing ferrate, wherein the fiber comprises
a. one or more ferrate compounds, wherein the ferrate compound is selected from a group consisting of metal ferrate(V) compounds, metal ferrate(VI) compounds, or mixtures thereof; and
b. one or more nonaqueous polymers.
